(19) United States
(12) Patent Application Publication
(10) Pub. No.: US 2024/0280564 A1
(43) Pub. Date: Aug. 22, 2024

(54) ASSAY FOR EXTRINSIC INHIBITION

(71) Applicants: The J. David Gladstone Institutes, a testamentary trust established under the Will of J. David Glads, San Francisco, CA (US); The Regents of The University of California, Oakland, CA (US)

(72) Inventors: Katerina Akassoglou, San Francisco, CA (US); Mark Petersen, San Francisco, CA (US); Anke Meyer-Franke, Menlo Park, CA (US)

(21) Appl. No.: 18/566,782

(22) PCT Filed: Jun. 3, 2022
(86) PCT No.: PCT/US2022/032172

§ 371 Date: Dec. 4, 2023

Related U.S. Application Data
(60) Provisional application No. 63/197,242, filed on Jun. 4, 2021.

(51) Int. Cl. G01N 33/50; C12N 5/00; C12N 5/079

(52) U.S. Cl. CPC G01N 33/5058; C12N 5/0018; C12N 5/0622; G01N 33/5026; G01N 33/5073; C12N 2501/13; C12N 2501/135; C12N 2506/08; G01N 2500/10

(57) ABSTRACT

A high-throughput, high-content assay to screen for an agent which overcomes remyelination inhibition by an extrinsic inhibitor.

### CROSS-REFERENCE TO RELATED APPLICATIONS

This application claims the benefit of the filing date of U.S. application No. 63/197,242, filed on Jun. 4, 2021, the disclosure of which is incorporated by reference herein.

### GOVERNMENT FUNDING

This invention was made with Government support under W81XWH-17-1-0211 awarded by the ARMY/MRMC and Government support under R35 NS097976 awarded by the National Institutes of Health. The Government has certain rights in the invention.

### INCORPORATION BY REFERENCE OF SEQUENCE LISTING PROVIDED AS A TEXT FILE

A Sequence Listing is provided herewith as a text file, “2244002.txt” created on Jun. 2, 2022 and having a size of 693 bytes. The contents of the text file are incorporated by reference herein in their entirety.

### BACKGROUND OF THE INVENTION

Extrinsic inhibitors at sites of blood-brain barrier (BBB) disruption and neurovascular damage contribute to remyelination failure in neurological diseases. However, therapies to overcome extrinsic inhibition of remyelination are not widely available and the dynamics of glial progenitor niche remodeling at site of neurovascular dysfunction are largely unknown.

### SUMMARY OF THE INVENTION

Provide herein is an assay/screen (named OPC-X) to identify compounds that promote remyelination in the presence of extrinsic inhibitors, as it was shown that promyelinating drugs did not rescue extrinsic inhibition of remyelination by fibrinogen.

One embodiment provides a high-throughput, high-content assay to screen for an agent which overcomes remyelination inhibition by an extrinsic inhibitor comprising: a) contacting oligodendrocyte progenitor cells (OPCs) with an extrinsic inhibitor and a test agent and b) obtaining two readouts in a single assay to detect/quantify the presence of: 1) MBP+ myelinating oligodendrocytes (OLs) and 2) GFAP+ astrocytes, wherein an increase in OLs and a decrease in GFAP+ astrocytes as compared to a control OPCs only contacted with the extrinsic inhibitor, indicates the agent overcame inhibition of remyelination by an extrinsic inhibitor.

In one embodiment, the extrinsic inhibitor is an antibody, a compound, a small molecule, a peptide and/or a nucleic acid. In another embodiment, the extrinsic inhibitor is an inflammatory molecule. In one embodiment, the extrinsic inhibitor is fibrinogen. In one embodiment, fibrinogen is present at a physiological level. In another embodiment, fibrinogen is added at a concentration of least, and including, 2.5 mg/ml.

In one embodiment, the OPCs are primary OPCs. In one embodiment, the primary OPCs are cultured in proliferation medium for 1-6 days prior to a). In another embodiment, the proliferation medium contains PDGF-AA and NT3. In one embodiment, the OPCs are detached from culture dish proteolytically and/or collagenolytically and then plated into fresh culture dishes. In another embodiment, the OPCs are plated at 5×10³ cells/well of a 96 well plate or 1×10³ cells/well of a 384 well plate prior to a). In one embodiment, plated OPCs are cultured for up to 24 hours prior to a). In another embodiment, the OPCs are cultured in step a) for 1-6 days, including 1, 2, 3, 4, 5 or 6 days, so that the OPCs can differentiate prior to b).

In one embodiment, after a), the cells are contacted with antibodies against MBP (oligodendrocytes) and antibodies against GFAP (astrocytes). In one embodiment, the antibodies are labeled directed or indirectly with a detectable label and images of the cells with labels are obtained, such as automated images are obtained. In one embodiment, at least about 80% of the culture vessel (e.g., well) is imaged. In another embodiment, automated quantification of MBP+ and GFAP+ is employed.

One embodiment provides a high-throughput, high-content assay to screen for an agent which overcomes inhibition of extrinsic inhibitor comprising: a) contacting a cell with an extrinsic inhibitor and a test agent and b) detecting and/or quantifying the cells response to the test agent, wherein a response by the cells that is different as compared to a control in which the cells are only contacted with the extrinsic inhibitor, indicates the agent was able to overcome the extrinsic inhibitor. In one embodiment, the extrinsic inhibitor is selected from the group consisting of chondroitin sulfate proteoglycan, hyaluronan, fibronectin aggregate, myelin debris, inflammatory cytokine (e.g., soluble TNF-alpha or Interferon-gamma), bone morphogenetic protein, endothelin-1, semaphorin, environmental toxin and alcohol, tobacco or illicit or recreational drugs. In another embodiment, the cell is selected from the group consisting of stem or progenitor cells including neural stem and/or progenitor cells (adult and/or fetal/neonatal), radial glial cells (adult and/or fetal/neonatal), cerebellar granule neuron progenitor cells, neural crest stem/progenitor cells, vascular/endothelial stem/progenitor cells, organ stem/progenitor cells (e.g., cardiac, liver, lung, kidney, skeletal muscle, skin, bone, retinal), mesenchymal stem/progenitor cells, placental stem/progenitor cells, embryonic stem cells, induced pluripotent stem cells (or cells derived from ESCs/iPSCs), and cancer/tumor-associated cells/stem cells. In one embodiment, the neural progenitors are oligodendrocyte progenitor cells (OPCs).

### BRIEF DESCRIPTION OF THE DRAWINGS

FIGS. 1′A-1′D. provides experimental and data for Example 1.

FIGS. 1A-1G. NG2 cells cluster perivascularly at sites of fibrinogen deposition and limited remyelination in chronic neuroinflammation. A, In vivo 2P maximum intensity projection images of microglia (green), NG2 cells (red) and the vasculature (blue, 70 kDa Oregon Green Dextran) in NG2-CreER^{TM}:Rosa^{tdTomato/+}:Cx3cr1^{GFP/+} age-matched healthy control mice, at the peak of clinical signs (peak EAE, mean score 3) and at chronic EAE (mean clinical score 2.1). Images shown are from mice on days 17 (peak) and 35 (chronic) after the induction of EAE. An NG2^{tdTomato+} pericyte in the control condition is depicted with a white arrow. Scale bar, 50 μm. Quantification of NG2 cell and microglial clusters in control (n=4 mice), peak (n=5 mice) and chronic (n=6 mice) EAE. Values are mean±s.e.m., *p<0.05, n.s. not significant, (two-way ANOVA with Bonferroni's multiple comparisons test). B, Microscopy of spinal cord sections from unimmunized healthy mice (control) and MOG₃₅₋₅₅-EAE mice at peak and chronic stages of disease immunostained for fibrinogen (green). Nuclei are stained with 4′,6-diamidino-2-phenylindole (DAPI, blue). Scale bar, 100 μm. Quantification of dextran leakage in spinal cord of unimmunized, healthy mice (control) (n=4 mice) and MOG35-55-EAE mice at peak (n=5 mice) and chronic (n=6 mice) stages of disease. Values are mean±sem., *p<0.05 (one-way ANOVA with Tukey's multiple comparisons test). Quantification of fibrinogen immunoreactivity in spinal cord of unimmunized healthy mice (control) and MOG₃₅₋₅₅-EAE mice at peak and chronic stages of disease (n=3 mice per group). Values are mean±sem., **p<0.01, ***p<0.001 (one-way ANOVA with Tukey's multiple comparisons test). C, Microscopy of ventral spinal cord sections of NG2-CreER^{TM}:Rosa^{tdTomato/+}:Cx3cr1^{GFP/+} mice at chronic EAE immunostained for fibrinogen (green). Scale bar, 50 μm. Quantification of fibrinogen immunopositivity in areas of NG2 clusters and areas without clusters (n=5 mice). Values are mean±s.e.m., **p<0.01 (two-tailed Mann-Whitney test). D, In vivo 2P maximum intensity projection images of myelin (green) in NG2-CreER^{TM}:Rosa^{tdTomato/+}:Cx3cr1^{GFP/+} mice at chronic EAE in areas of NG2 clusters and areas without clusters. Boxed areas are shown in top right insets to only depict myelin labeling. Scale bar, 20 μm. Quantification of myelin circularity at chronic EAE in areas of NG2 clusters and areas without clusters (n=5 mice). Values are mean±s.e.m., **p<0.01 (two-tailed Mann-Whitney test). A value of 1.0 indicates a perfect circle (as seen in degenerating myelin in longitudinal sections); as the value approaches 0.0, it indicates an increasingly noncircular, linear shape (longitudinal section of normal myelinated fiber). E, ROI tracking workflow for the co-registration of 2P and SBEM volumes. F-G, Representative co-related SBEM images from n=3 ROIs from 2 different mice. Fi, CNS parenchyma in areas of NG2 clusters shows an inflamed spinal cord vessel with activated endothelial cells (green asterisk), attachment of a leukocyte to the endothelium (black arrowhead) and perivascular lesions with dominant demyelination (red boxed area) and sparse remyelination (blue boxed area). Scale bar, 20 μm. Fii, red boxed area is shown at higher magnification. Red arrows depict demyelinated axons. Scale bar, 10 μm. Fiii, blue boxed area is shown at higher magnification. Blue arrows depict remyelinated axons. Scale bar, 10 μm. Fiv, Correlated SBEM within the CNS parenchyma in an area without NG2 clusters. Black arrows depict normal myelinated axons. Scale bar, 10 μm. Gi, Representative SBEM from another ROI in an area of NG2 cluster shows a vein with perivascular demyelination, gliosis (red dotted area) and some limited remyelination (blue boxed area). The area of gliosis contains an infiltrating macrophage (M) and an astrocyte (A). Distal areas have normal myelinated axons depicted with black arrows. Scale Bar, 10 μm. Gii, blue boxed area is shown at higher magnification. Blue arrows depict remyelinated axons. Black arrowheads depict NG2 cells. Scale Bar, 5 μm.

FIGS. 2A-2F. RNA-seq analysis of NG2 cells in EAE reveals suppression of anticoagulation pathways. Data are from n=3 mice per group (A-D). A, Volcano plot of DEGs from RNA-seq analysis of NG2 lineage cells from MOG₃₅₋₅₅-EAE or healthy mice. Circles depict genes significantly downregulated (blue; log 2 fold change<−1; FDR<0.05) or upregulated (red; log 2 fold change>1; FDR<0.05) in EAE compared to healthy mice. B, Heat map of data from A. Genes were clustered by HOPACH unsupervised clustering analysis (Clusters 1-9). Expression values were log normalized, row centered and depicted as z-score. Significant GO terms and example genes are shown for each cluster. FDR<0.05; Benjamini-Hochberg correction. C, Visualization of co-expression GO term networks downregulated (blue nodes) or upregulated (red nodes) in NG2 cells from EAE compared to healthy mice. Gene set size and co-expression overlap (key) was determined by GSEA, p<0.05. D, Enrichment plot for the gene sets “Negative regulation of coagulation” and “Regulation of cell junction assembly” determined by GSEA of RNA-seq data of NG2 cells from EAE or healthy mice. X-axis depicts gene rank in dataset. NES, normalized enrichment score. E-F, Representative histograms of surface labeled TFPI and quantification of TFPI+ cells in PDGFRα+ OPC (E) or PDGFRβ+ pericyte (F) populations from healthy and EAE mice. Data are from n=5 per group (mean±s.e.m.) **p<0.01, n.s. not significant (two-tailed Mann-Whitney test).

FIGS. 3A-3G. Promyelinating compounds do not overcome fibrinogen extrinsic inhibition of OPC differentiation. A, Workflow for medium throughput, OPC-X screen of promyelinating drugs in the presence of fibrinogen. B-C, Immunofluorescence for MBP (green) and GFAP (red) in primary rat OPCs treated with fibrinogen and myelin-promoting drugs or vehicle control (dimethylsulfoxide, DMSO) as indicated. Nuclei are stained with Hoechst dye (blue). Representative images from n=3 independent experiments. Scale bar, 100 μm. D-E, Quantification of percentage of total cells MBP+ or GFAP+ from automated image acquisition and quantification. Data are mean±s.e.m. from n=3 independent experiments. ****p<0.0001 (one-way ANOVA with Dunnett's multiple comparisons test). F, Phospho-SMAD1/5 (P-SMAD1/5) and ID2 protein levels in control or fibrinogen-treated primary rat OPCs in the presence of DMH1 or clemastine. Values are mean of n=3 independent experiments. G, Immunofluorescence for MBP (green) and GFAP (red) in primary rat OPCs treated with fibrinogen and LDN-212854 (0.18 μM) or vehicle control (DMSO) for three days. Nuclei are stained with Hoechst dye (blue). Representative images from n=3 independent experiments. Scale bar, 100 μm. H, Quantification of percentage of total MBP+ or GFAP+ cells from automated image acquisition and quantification. Data are mean±s.e.m. from n=3 independent experiments. *p<0.05, **p<0.01, ***p<0.001, ****p<0.0001 (matched oneway ANOVA with Dunnett's multiple comparisons test).

FIGS. 4A-4E. Therapeutic effects of type I BMP receptor inhibition in chronic neuroinflammation. A, Clinical scores for MOG₃₅₋₅₅-EAE mice treated with LDN-212854 or saline (key) for 14 days starting at peak disease. Data are from n=6 mice (EAE+ LDN-212854) and n=5 mice (EAE+saline), mean±s.e.m., *p<0.05, (two-tailed permutation test). B, Microscopy of spinal cord sections from MOG₃₅₋₅₅-EAE mice treated with saline (left panel) or LDN-212854 (right panel) immunostained for MBP to visualize myelin (green) and fibrinogen (red). Dashed line demarcates demyelinated white matter. Scale bar, 50 μm. Data are from n=5 mice per group, mean±s.e.m., **p<0.01 (two-tailed Mann-Whitney test). C, Clinical scores for NOD-MOG₃₅₋₅₅ EAE mice treated with LDN-212854 or saline (key) for 30 days. Data are from n=8 mice (EAE+ LDN-212854) and n=7 mice (EAE+saline), mean±s.e.m., *p<0.05, (Welch two-sample t-test comparing the group means of maximum scores, Saline=2.36, LDN-212854=1.75). D, Microscopy of spinal cord sections from NOD-MOG₃₅₋₅₅ EAE mice treated with saline (left panel) or LDN-212854 (right panel) with darkfield microscopy used to visualize myelin (green) and immunostained for fibrinogen (red). Dashed line demarcates demyelinated white matter. Scale bar, 100 μm. Data are from n=6 mice per group, mean±s.e.m., *p<0.05 **p<0.01 (two-tailed Mann-Whitney test). E, In vivo 2P maximum intensity projection images of NG2 cells (red) and the vasculature (blue, 70 kDa Oregon Green Dextran) in NG2-CreER^{TM}:Rosa^{tdTomato/+} mice at chronic EAE treated with saline (left panel) and LDN-212854 (right panel). Scale bar, 50 μm. Data are from n=6 (EAE+ LDN-212854) and n=5 (EAE+saline), mean±s.e.m, *p<0.05 (two-tailed unpaired t-test). F, In vivo 2P maximum intensity projection images of NG2 cells (red) and myelin (green, MitoTracker) in NG2-CreER^{TM}:Rosa^{tdTomato/+} mice at chronic EAE treated with saline (left panel) and LDN-212854 (right panel). Scale bar, 20 μm. Data are from n=5 (EAE+ LDN-212854) and n=4 (EAE+saline), mean±s.e.m., *p<0.05 (two-tailed Mann-Whitney test). Myelin damage was quantified with myelin circularity where a value of 1.0 indicates a perfect circle; as the value approaches 0.0, it indicates an increasingly noncircular shape, linear shape. G, Microscopy of spinal cord sections from NG2-CreER^{TM}:Rosa^{tdTomato/+} MOG₃₅₋₅₅-EAE mice after 14 day treatment of saline (left panel) or LDN-212854 (right panel). NG2 cells (red) and immunostaining for ID2 (green). Nuclei are stained with 4′,6-diamidino-2-phenylindole (DAPI, blue). Scale bar, 25 μm. Data are from n=6 (EAE+ LDN-212854) and n=5 (EAE+saline), mean±s.e.m., **p<0.01 (twotailed Mann-Whitney test). H, Fate mapping of tdTomato⁺ OPC-derived cells using microscopy of spinal cord sections from NG2-CreER^{TM}:Rosa^{tdTomato/+} MOG₃₅₋₅₅-EAE mice after 14 day treatment of LDN-212854 or saline. NG2^{tdTomato+} cells (red) and immunostaining for the mature OL marker GST-pi (green, top panel) or the astrocyte marker GFAP (green, bottom panel). Scale bar, 50 μm (top panel) and 20 μm (bottom panel). Data are from n=6 (EAE+ LDN-212854) and n=5 (EAE+saline), mean±s.e.m., **p<0.01 (two-tailed Mann-Whitney test).

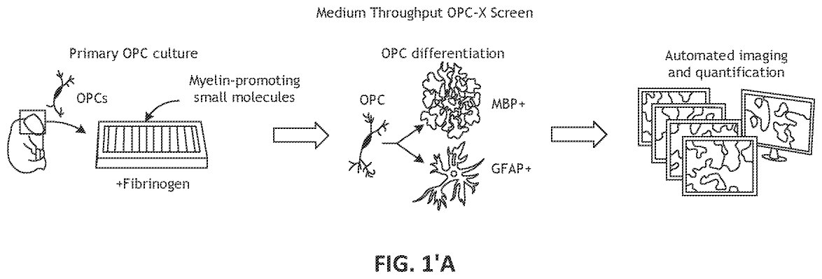

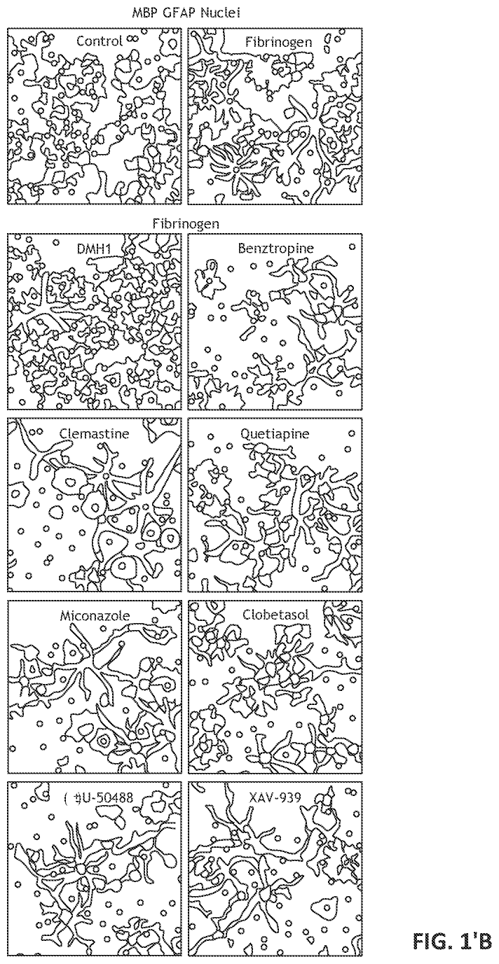

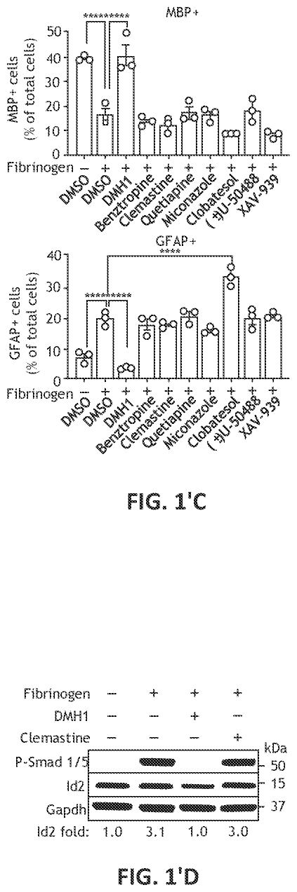

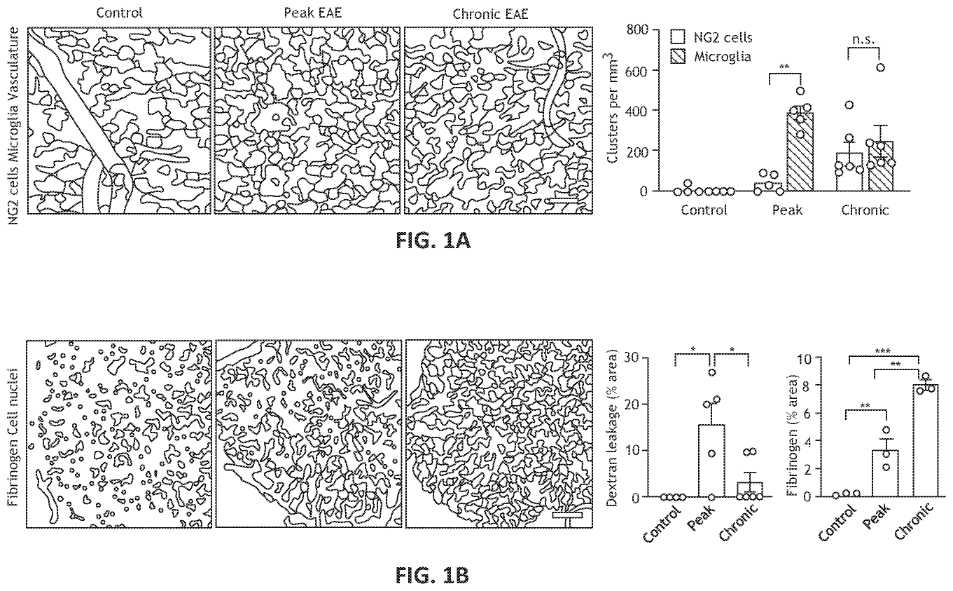

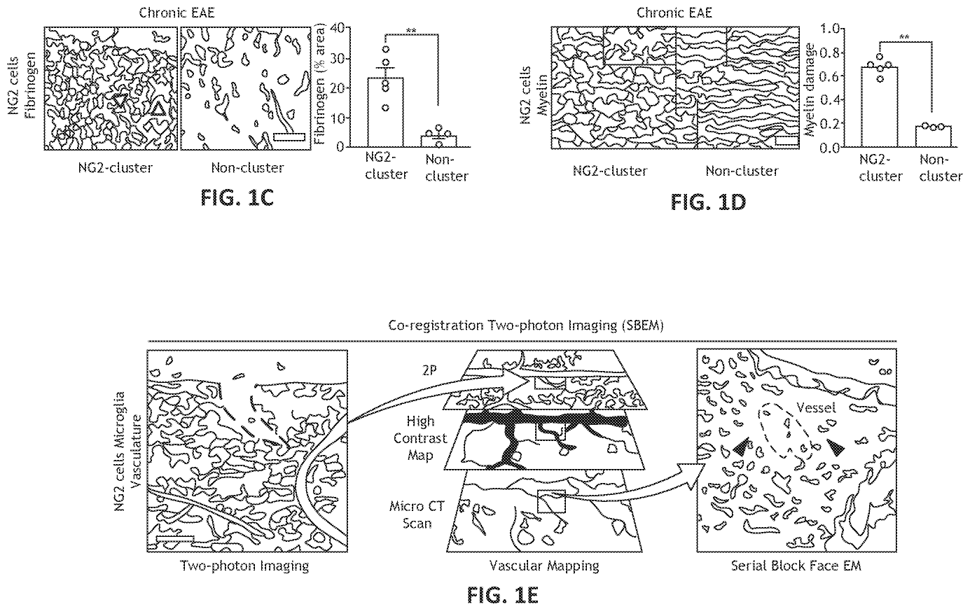

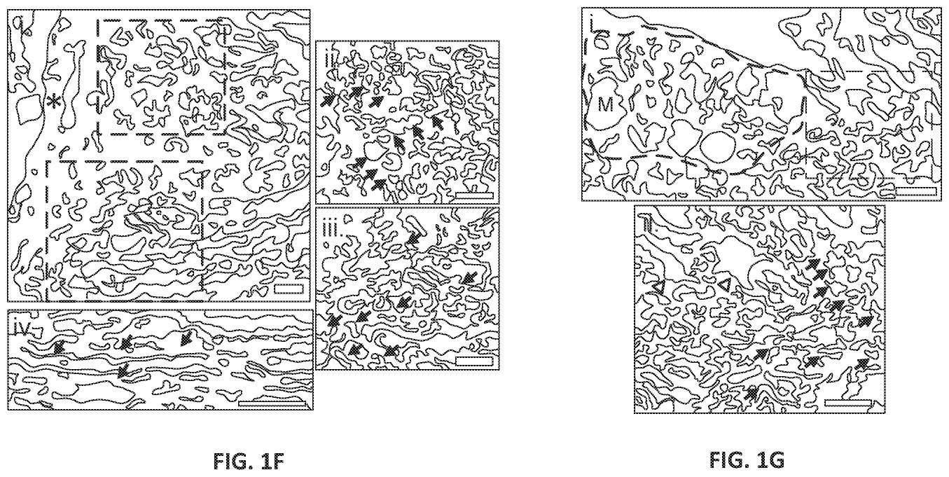

Supp FIG. 1. Workflow for in vivo 2P imaging and bulk RNA-seq analysis of NG2-lineage cells and microglia in NG2creER^{TM}:Rosa^{tdTomato/+}:Cx3cr1^{GFP/+} mice in MOG₃₅₋₅₅-EAE.

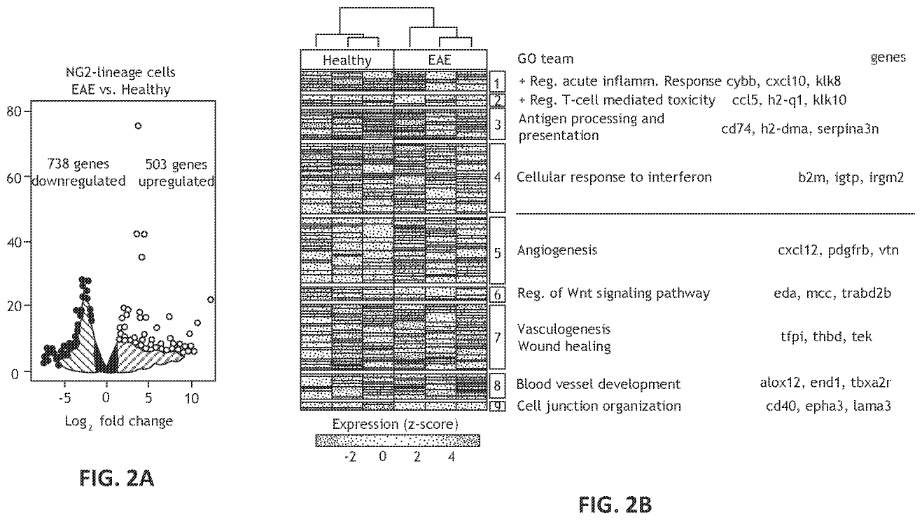

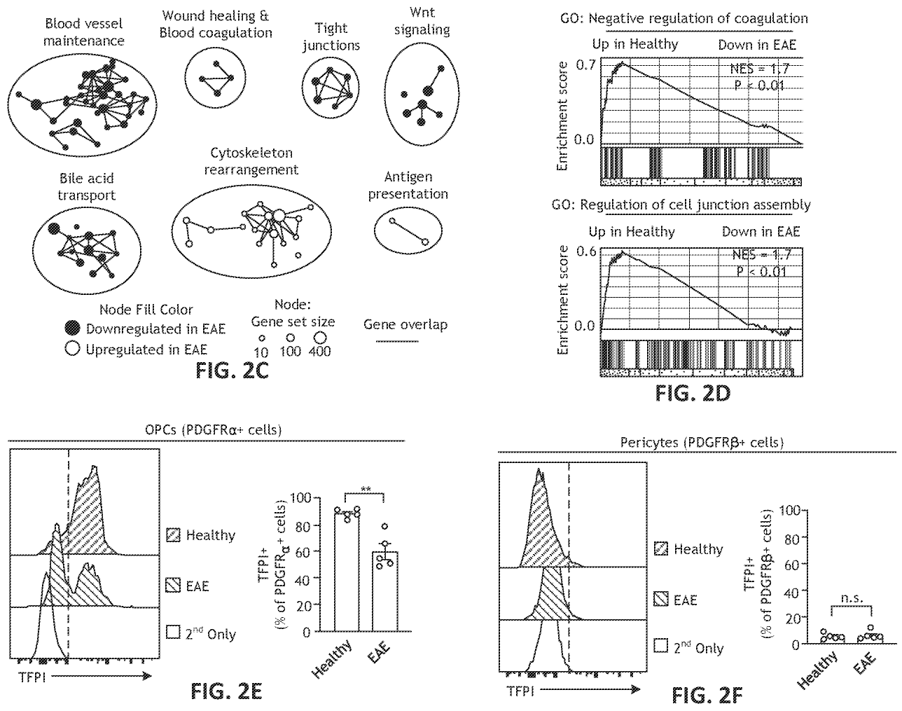

Supp FIGS. 2A-2C. In vivo 2P imaging of NG2 cells and microglia at the neurovascular interface at different stages of EAE. In vivo 2P maximum intensity projection images of NG2 cells (red, top panel), microglia (green, bottom panel) and the vasculature (blue, 70 kDa Oregon Green Dextran) in NG2creER^{TM}:Rosa^{tdTomato/+}:Cx3cr1^{GFP/+} age-matched healthy control mice, at the peak of clinical signs (peak EAE, mean score 3) and at chronic EAE (mean clinical score 2.1). Scale bar, 100 μm. Quantification of co-localization of NG2 clusters and microglial clusters at peak (n=5 mice) and chronic (n=6 mice) EAE. Values are mean±s.e.m., **p<0.01 (two-tailed Mann-Whitney test). B, In vivo 2P maximum intensity projection images of NG2 cells (red) and the vasculature (blue, 70 kDa Oregon Green Dextran) in NG2creER^{TM}:Rosa^{tdTomato/+}:Cx3cr1^{GFP/+} age-matched healthy control mice, at the peak of clinical signs (peak EAE, mean score 3) and at chronic EAE (mean clinical score 2.1). Scale bar, 50 μm. Quantification of the distance of NG2 clusters from the nearest blood vessel at chronic EAE (data from 45 clusters in 6 mice). An NG2^{tdTomato+} pericyte in the control condition is depicted with a white arrow. C, In vivo 2P maximum intensity projections of tdTomato⁺ (red) pericytes (left panel) and OL-lineage cell in relation to the vasculature (blue, 70 kDa Oregon Green Dextran) in the spinal cord parenchyma of NG2-CreER^{TM}:Rosa^{tdTomato/+}:Cx3cr1^{GFP/+} mice. Scale bar, 20 μm.

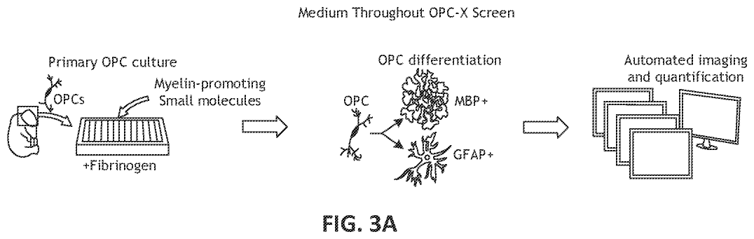

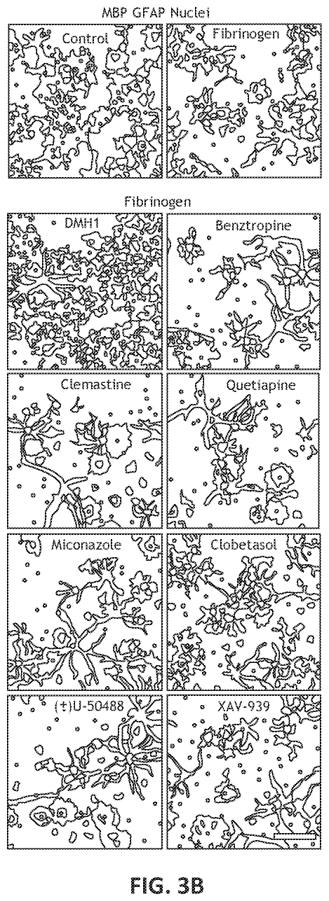

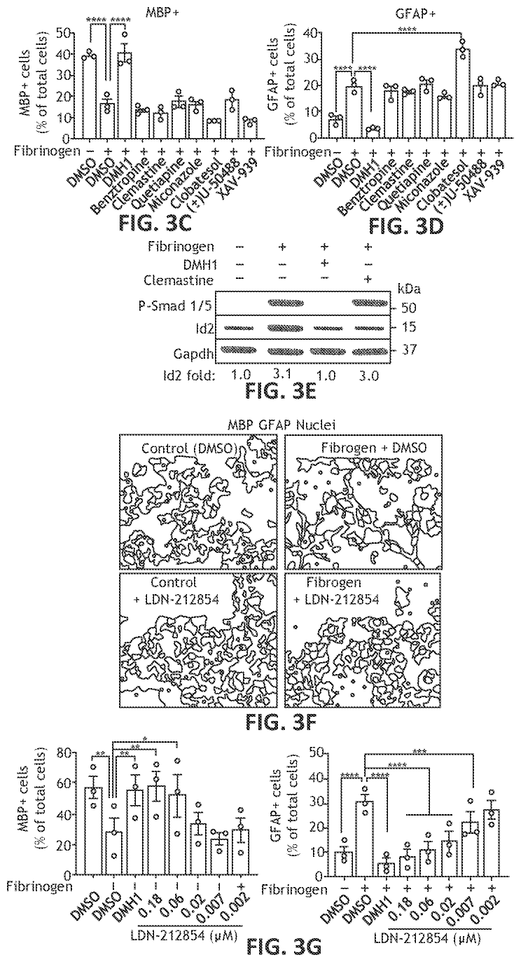

Supp FIGS. 3A-3C. Endothelial activation at different stages of EAE. A, Microscopy of ventral spinal cord sections of NG2-CreER^{TM}:Rosa^{tdTomato/+} mice in control, peak EAE and chronic EAE immunostained for VCAM-1. Red arrows depict vascular VCAM-1 expression; red asterisks depict diffuse VCAM-1 positivity. Quantification of VCAM-1 immunoreactivity in ventral spinal cord in control, peak EAE and chronic EAE. Scale bar, 50 μm. Values are mean±s.e.m., **p<0.05 (one-way ANOVA with Dunnett's multiple comparisons test). B, Microscopy of ventral spinal cord sections of NG2-CreER^{TM}:Rosa^{tdTomato/+} mice in control, peak EAE and chronic EAE immunostained for PLVAP. Red arrows depict vascular PLVAP expression; red asterisks depict diffuse PLVAP positivity. Scale bar, 50 μm. Quantification of PLVAP+ vessels in ventral spinal cord in control, peak EAE and chronic EAE. Values are mean±s.e.m., *p<0.05 (one-way ANOVA with Tukey's multiple comparisons test). C, CNS parenchyma in areas of NG2 clusters shows an inflamed spinal cord vessel with activated endothelial cells. Depicted here are activated endothelia (black arrows) which are thicker compared to the very thin endothelia in normal BBB vessels. These activated endothelia form small protrusions or processes (red arrow), which make contacts with leukocytes (black arrowhead) within the vessel.

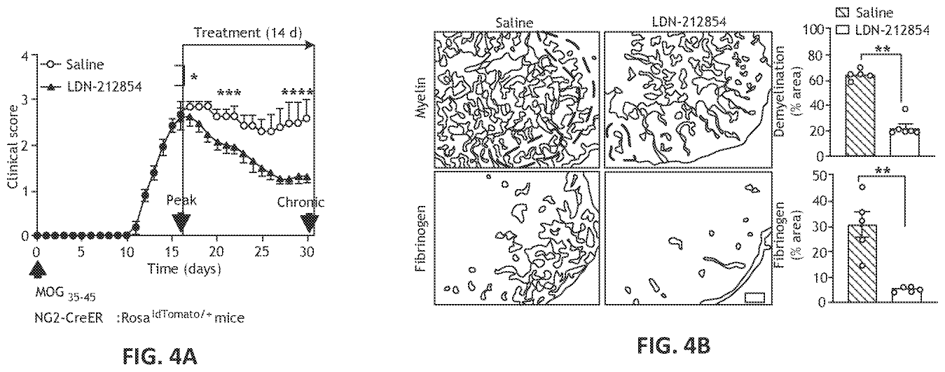

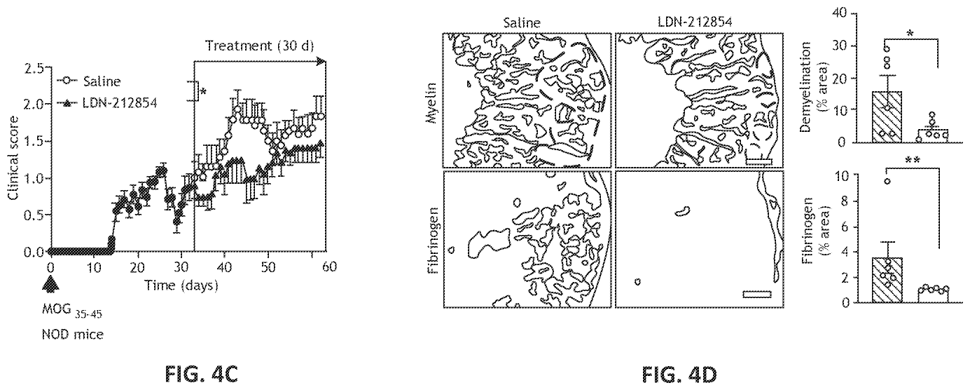

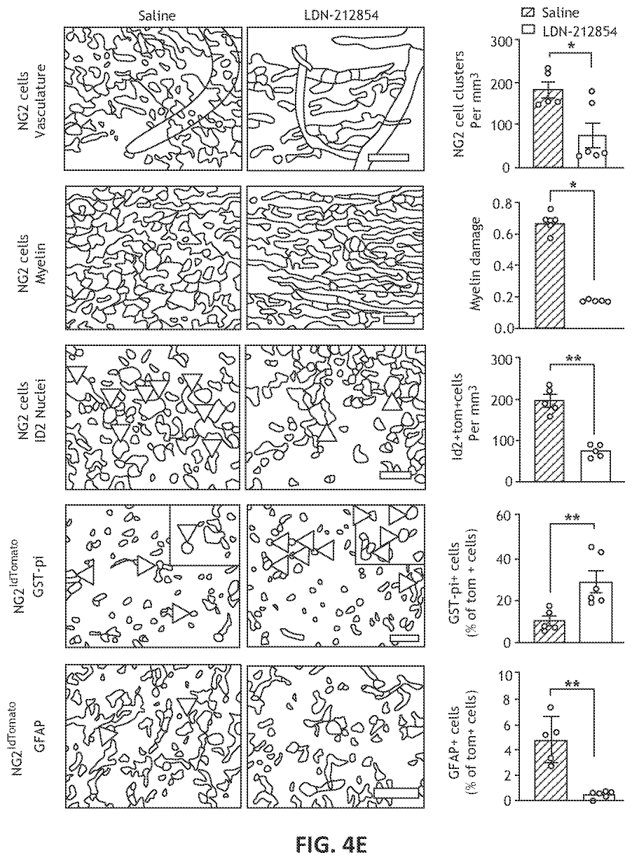

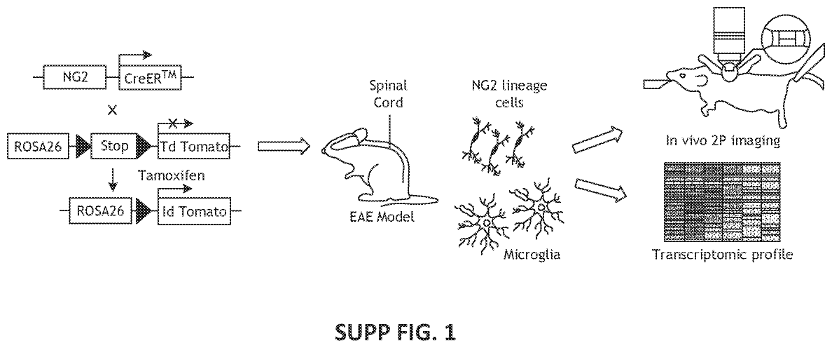

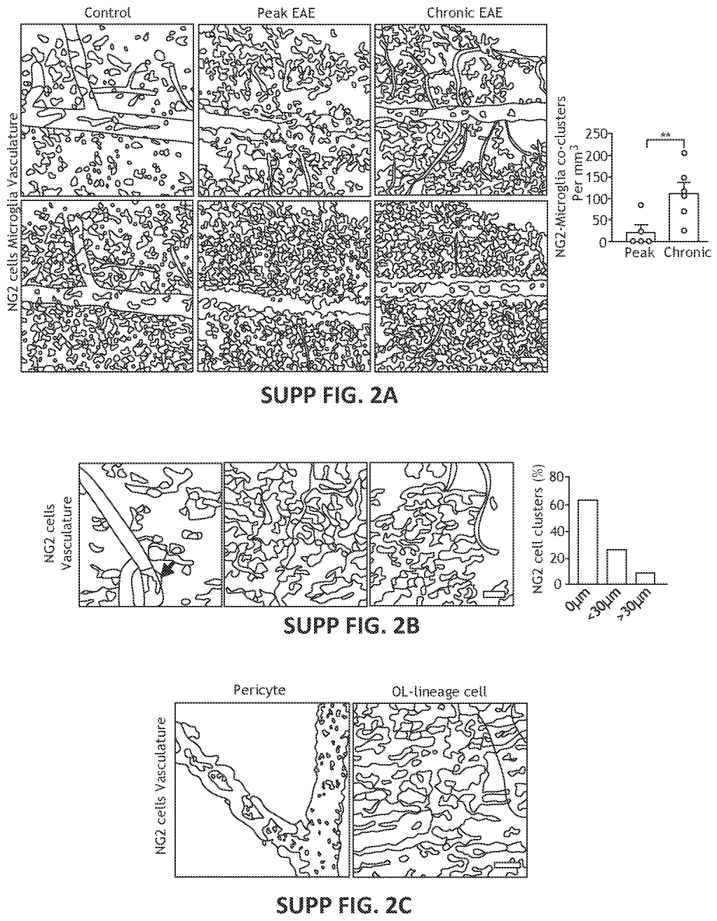

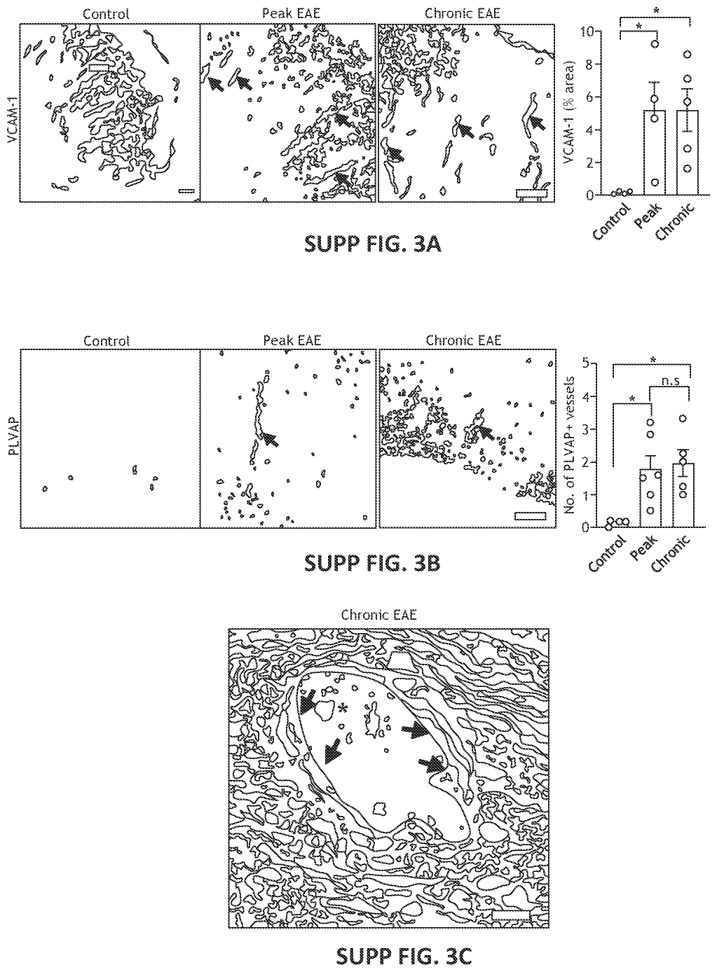

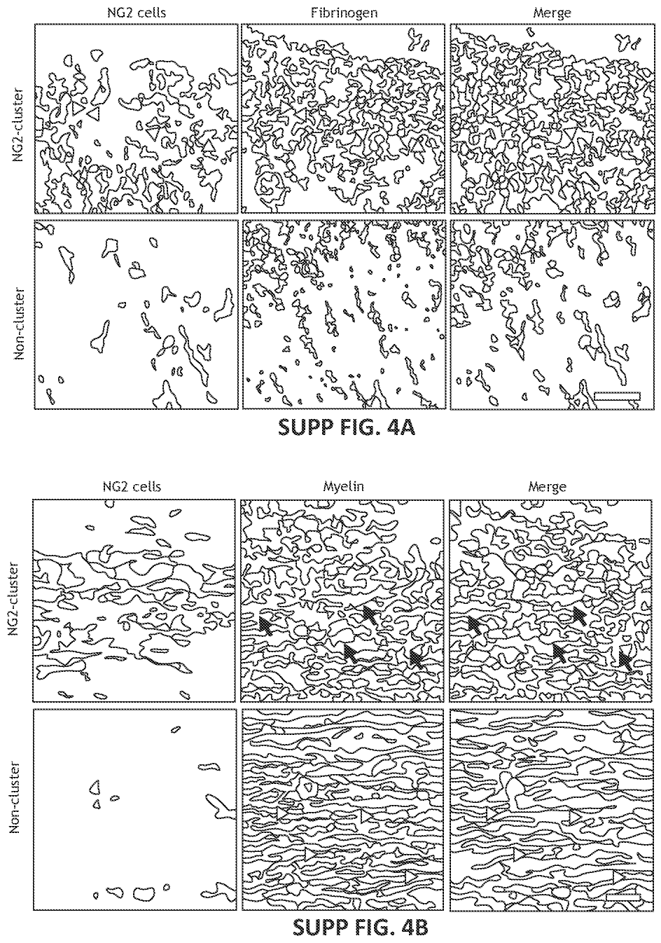

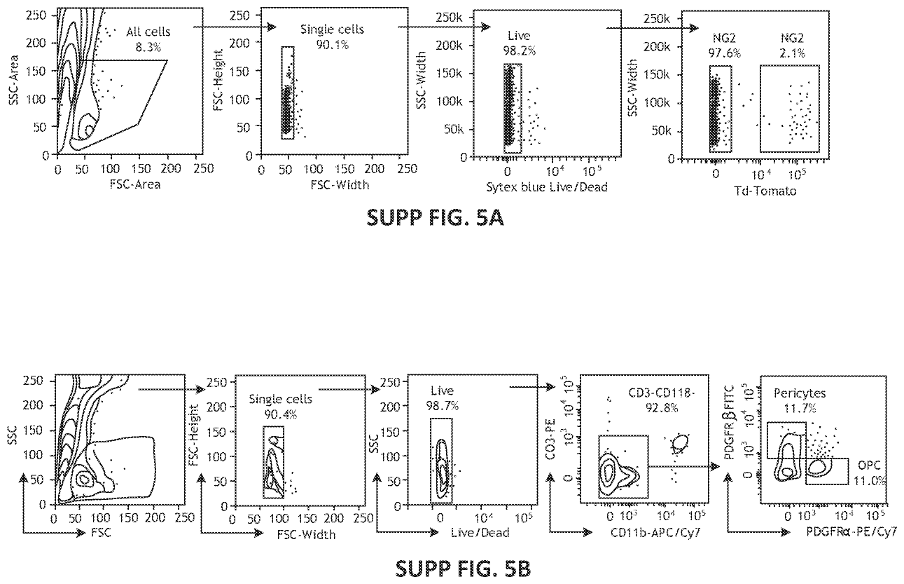

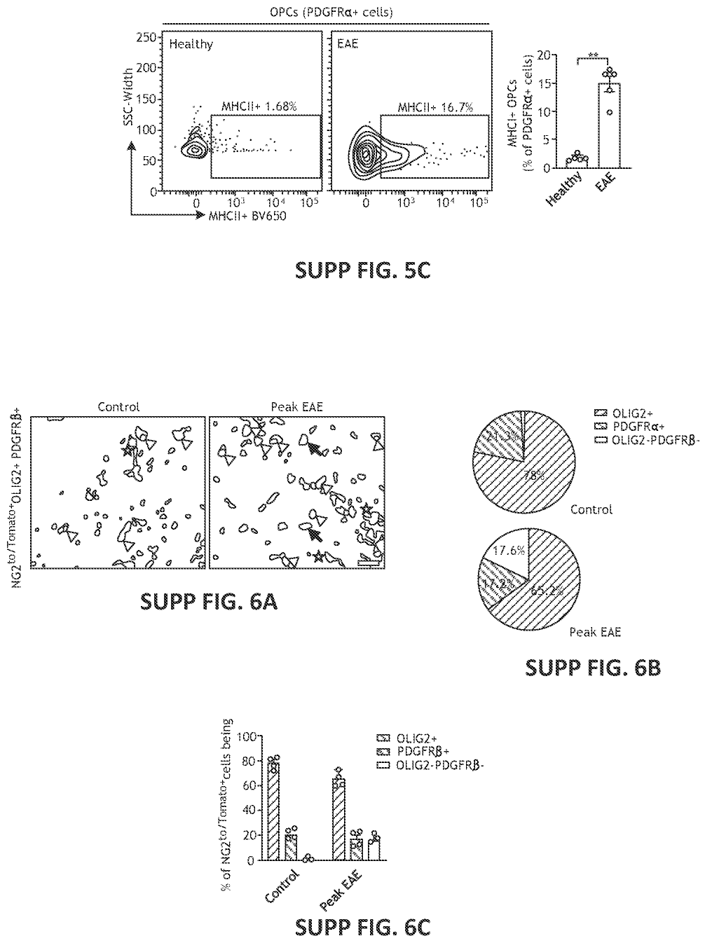

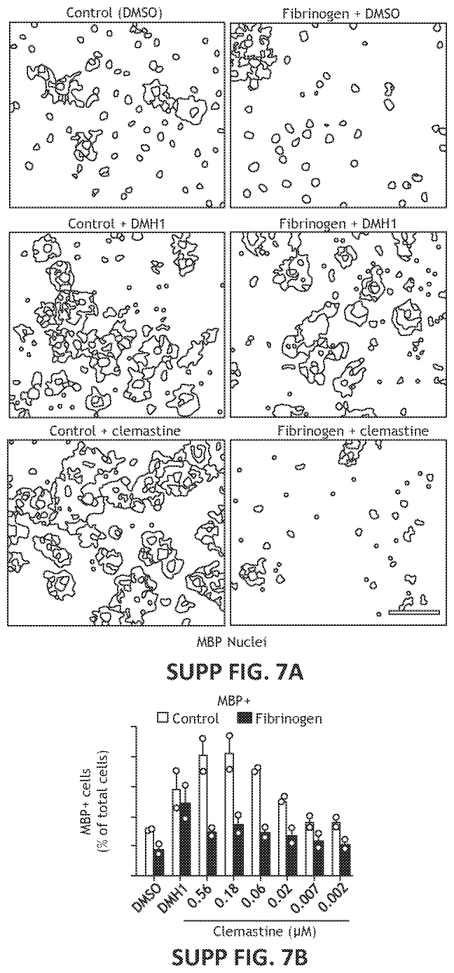

Supp FIGS. 4A-4B. NG2 cell clusters associated with fibrinogen deposition and myelin disruption at chronic EAE. A, Microscopy of ventral spinal cord sections of NG2-CreER^{TM}:Rosa^{tdTomato/+}:Cx3cr1^{GFP/+} mice at chronic EAE immunostained for fibrinogen (green). NG2^{tdTomato+} cells (red) cluster at sites of fibrinogen deposition, depicted here in the merge channel with yellow ROIs (white arrowheads). Scale bar, 50 μm. B, In vivo 2P maximum intensity projection images of NG2^{tdTomato+} cells (red) and myelin (green) in NG2-CreER^{TM}:Rosa^{tdTomato/+}:Cx3cr1^{GFP/+} mice at chronic EAE in areas of NG2 cell clusters and areas without clusters. It is important to note that myelin sheathes are labeled with Mito Tracker Deep Red far red-fluorescent dye (abs/em˜644/665 nm), pseudocolored here in green. Disrupted myelin or myelin blebs are shown here with white arrows in areas of NG2 cell clusters and normal-appearing myelin is depicted with white arrowheads in non-cluster areas. Scale bar, 20 μm.

Supp FIGS. 5A-5C. FACS isolation of NG2 cells. A, Representative flow cytometry plots of the gating strategy for NG2^{tdTomato+} cells from the spinal cord of EAE (n=3) and healthy control mice (n=3) for bulk RNA-seq. B, Representative flow cytometry plots of the gating strategy for PDGFRα⁺ and PDGFRβ⁺ cells from the spinal cord of chronic EAE (n=5) and healthy control mice (n=5) for cell surface staining. C, Representative flow cytometry contour plot and quantification of surface MHCII in live PDGFRα⁺ cells. Data are from n=5 per group (mean±s.e.m.) **p<0.01, (two-tailed Mann-Whitney test). Percent of cell population is listed above gate (A-C).

Supp FIGS. 6A-6C. Ratio of oligodendroglial lineage cells and pericytes amongst NG2^{tdTomato+} cells in control and Peak EAE. A, Microscopy of ventral spinal cord sections of NG2-CreER^{TM}:Rosa^{tdTomato/+} mice in control and at peak EAE with NG2^{tdTomato+} cells (red) immunostained for OLIG2 (green) and PDGFRB (stained in far red channel, pseudocolored here in blue). NG2^{tdTomato+} OLIG2⁺ cells are depicted with white arrowheads; NG2^{tdTomato+} PDGFRβ⁺ cells are depicted with white asterisks. NG2^{tdTomato+} OLIG2⁻ PDGFRβ⁻ cells are depicted with white arrows. Scale bar, 20 μm. B-C, Quantifications of the percentage of NG2^{tdTomato+} cells that are OLIG2⁺ and PDGFRβ⁺ in control and at peak EAE.

Supp FIGS. 7A-7B. Effect of clemastine on primary OPCs in the presence of fibrinogen. A, Immunofluorescence for MBP (green) in primary rat OPCs treated with fibrinogen and clemastine (0.56 μM), DMH1 (1 μM), or vehicle control (dimethylsulfoxide, DMSO) for three days in differentiation media without T3 or growth factors. Nuclei are stained with Hoechst dye (blue). Representative images from n=2 independent experiments. Scale bar, 100 μm. B, Quantification of percentage of total cells MBP+ from automated image acquisition and quantification. Data are mean±s.e.m. from n=2 independent experiments.

### DETAILED DESCRIPTION OF THE INVENTION

The practice of the methods and compositions described herein may employ, unless otherwise indicated, conventional techniques of pharmaceutical chemistry, drug formulation techniques, dosage regimes, molecular biology and biochemistry, all of which are within the skill of those who practice in the art. Specific illustrations of suitable techniques can be had by reference to the examples herein.

In the following description, numerous specific details are set forth to provide a more thorough understanding of the present invention. However, it will be apparent to one of skill in the art that the present invention may be practiced without one or more of these specific details. In other instances, features and procedures well known/available to those skilled in the art have not been described in order to avoid obscuring the invention.

### Abbreviations

2P=two-photon; BBB=blood-brain barrier; BMP=bone morphogenetic protein; CSPG=chondroitin sulfate proteoglycan; DEGs=differentially expressed genes; EAE=experimental autoimmune encephalomyelitis; GSEA=gene set enrichment analysis; GO=gene ontology; GST-pi=glutathione s-transferase-pi; MHC II=major histocompatibility complex class II; MOG=myelin oligodendrocyte glycoprotein; NOD=non-obese diabetic; OL=oligodendrocyte; OPC=oligodendrocyte progenitor cell; RNA-seq=RNA-sequencing; SBEM=serial block face electron microscopy; TFPI=tissue factor pathway inhibitor; TGF-β=transforming growth factor-beta.

### DESCRIPTION

Remyelination assays that consist of OPCs with a single readout of MBP+ oligodendrocytes are neither suitable to screen for extrinsic inhibitors of remyelination present in the lesion environment, nor for cell-fate switch to GFAP+ astrocytes.

By developing the screen (OPC-X) to identify compounds that promote remyelination in the presence of extrinsic inhibitors, it was shown promyelinating drugs did not rescue extrinsic inhibition of remyelination by fibrinogen. In contrast, bone morphogenetic protein (BMP) receptor blockade rescued the inhibitory fibrinogen effects and restored a promyelinating progenitor niche by promoting myelinating oligodendrocytes, while suppressing astrocyte cell fate with potent therapeutic effects in chronic models of multiple sclerosis. Thus, abortive OPC differentiation by fibrinogen is refractory to known promyelinating compounds, suggesting that blockade of the BMP signaling pathway may enhance remyelinating efficacy by overcoming extrinsic inhibition in neuroinflammatory lesions with vascular damage.

The assay can used for numerous purposes, including to screen antibodies, compounds, small molecules, peptides, etc. that a) overcome inhibition of remyelination by fibrin/fibrinogen b) overcome inhibition of remyelination by other extrinsic inhibitors, such as inflammatory molecules, cytokines, etc., c) inhibit the generation of fibrotic astrocytes, and d) inhibit the cell-fate switch of oligodendrocyte progenitor cells (OPCs) or other stem cells like neuronal precursor cells (NPCs) to astrocytes.

The assay provided herein provides several advantages, including, 1) screening and discovery of agents that can rescue disease-relevant inhibition of remyelination; 2) optimized for fibrin/fibrinogen as a disease-relevant extrinsic inhibitor; 3) can be adapted for use with other extrinsic inhibitors in addition to or in place of fibrin/fibrinogen and 4) allows for testing of both myelinating cells and astrocytes in a single assay.

In addition to or in place of fibrin/fibrinogen, other extrinsic inhibitors include, but are not limited to, chondroitin sulfate proteoglycans (Keough et al., 2016), hyaluronan (Srivastava et al., 2018), fibronectin aggregates (Stoffels et al., 2013), myelin debris (Kotter et al., 2006), inflammatory cytokines (e.g., soluble TNF-alpha (Karamita et al., 2017), Interferon-gamma (Kirby et al., 2019)), bone morphogenetic proteins (Mabie et al., 1997), endothelin-1 (Hammond et al., 2014), semaphorins (Syed et al., 2011), environmental toxins and alcohol/tobacco/illicit and/or recreational drugs (Forbes and Gallo, 2017).

Cells for use in the assay/screen described herein include, but are not limited to, neural stem/progenitor cells (adult and fetal/neonatal), radial glial cells (adult and fetal/neonatal), cerebellar granule neuron progenitor cells, neural crest stem/progenitor cells, vascular/endothelial stem/progenitor cells, organ stem/progenitor cells (Cardiac, Liver, Lung, Kidney, Skeletal Muscle, Skin, Bone, Retinal), mesenchymal stem/progenitor cells, placental stem/progenitor cells, embryonic stem cells and/or induced pluripotent stem cells (and cells derived from ESCs/iPSCs) and cancer/tumor-associated stem cells.

Such a screen/assay can be used to identify therapeutic agents/molecules for the treatment of numerous conditions/diseases, including, but not limited to, neurological diseases, such as those with BBB disruption and fibrin deposition (Petersen et al., 2018), Alzheimer disease, age-related dementia, traumatic brain and spinal cord injury, neonatal and preterm infant brain injury, subarachnoid/intraventricular hemorrhage, stroke, infection, amyotrophic lateral sclerosis, Parkinson disease, Huntington disease, HIV encephalitis, neuropsychiatric disorders such as schizophrenia and bipolar disease, cancer, atherosclerosis/cardiovascular disease, retinopathy/macular degeneration, chronic lung disease, peripheral autoimmune diseases (e.g., rheumatoid arthritis, colitis, or lupus), epithelial to mesenchymal transition (EMT) and multiple sclerosis (MS; e.g., drugs that overcome the fibrinogen-rich, inhibitory MS lesion environment would provide a desperately needed alternative therapeutic avenue for MS).

### Use of Test Agents

The assays of the invention are used to identify candidate therapeutic agents that inhibit extrinsic inhibition. This includes the testing of new agents as well as assays to test known compounds (including synthetic, recombinant or naturally occurring compounds) for their effect.

It is known in the pharmaceutical arts that binding affinity to a target and efficacy do not necessarily correlate, and that identification of cell-based activity changes conferred by a test agent is an improved functional predictor of therapeutic activity compared to agents identified merely by affinity, e.g., binding of agents to microglial receptors.

In certain aspects, the assays of the invention correlate with in vivo modulation of signaling through activated fibrin. Examples of cell-based assays for use with the present invention include, but are not limited to, high throughput binding screening; assays to measure cell activation, proliferation, differentiation, necrosis and/or apoptosis; flow cytometry assays; metabolic assays measuring labeling or turnover; phase and fluorescence microscopy; receptor phosphorylation and/or turnover; cell signaling assays; immunohistochemistry studies; reporter gene assays, and subcellular fractionation and localization.

Biochemical assays can also be used to correlate binding with efficacy in the cell-based assay methods of the invention. These include, but are not limited to, spectrophotometric assays, fluorometric assays, calorimetric assays, chemiluminescent assays, radiometric assays, chromatographic assays, colorimetric assays, and substrate specificity inhibitor kinase assays. Specific examples are luciferase assays, in which firefly luciferase protein catalyzes luciferin oxidation and light is generated in the reaction, and which is frequently used as a reporter gene for measuring promoter activity or transfection efficiency; electrophoresis; gas-liquid chromatography; and Forster resonance energy transfer (FRET).

To confirm the functional activity of a test agent, a therapeutically effective amount of a test agent of the invention may be administered to a subject (including an animal model of a neurological pathology) to confirm its in vivo activity following identification in an assay of the invention. By “therapeutically effective dose or amount” or “effective amount” is meant an amount of the test agent that, when administered, brings about a positive therapeutic response. In some embodiments of the invention, the therapeutically effective dose is in the range from about 0.1.mu.g/kg to about 100 mg/kg body weight, about 0.001 mg/kg to about 50 mg/kg, about 0.01 mg/kg to about 30 mg/kg, about 0.1 mg/kg to about 25 mg/kg, about 1 mg/kg to about 20 mg/kg, about 3 mg/kg to about 15 mg/kg, about 5 mg/kg to about 12 mg/kg, about 7 mg/kg to about 10 mg/kg or any range of value therein. It is recognized that the method of treatment may comprise a single administration of a therapeutically effective dose or multiple administrations of a therapeutically effective dose.

The test agent is administered to supply a desired therapeutic dose to promote a desired therapeutic response. By “desired therapeutic response” is intended an improvement in the condition or in the symptoms associated with the condition. Examples of routes of administration include intravenous, intraarterial, intracoronary, parenteral, subcutaneous, subdermal, subcutaneous, intraperitoneal, intraventricular infusion, infusion catheter, balloon catheter, bolus injection, direct application to tissue surfaces during surgery, or other convenient routes.

The test agents can be formulated in a unit dosage such as a solution, suspension or emulsion, in association with a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical. Sciences, a standard reference text in the field, which is incorporated herein by reference. Preferred examples of such carriers or diluents include, but are not limited to, water, saline, Ringer's solutions, dextrose solution, and 5% human serum albumin. The use of such media and agents for delivering cells is well known in the art. Except insofar as any conventional media or agent is incompatible with the cells or polypeptides provided herein, use thereof in the compositions is contemplated. Supplementary active compounds can also be incorporated into the test agents.

Solutions or suspensions used for such administration can include other components such as sterile diluents like water for dilution, saline solutions, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The composition can be enclosed in ampoules, disposable syringes or multiple dose vials made of glass or plastic.

Test agents suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). In all cases, the composition must be sterile and should be fluid to the extent possible. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents.

Prolonged absorption of the injectable compositions can be brought about by including in the composition an agent which delays absorption, for example, aluminum monostearate and gelatin. Sterile injectable solutions can be prepared by incorporating an active agent in the required amount in an appropriate solvent with a selected combination of ingredients, followed by filter sterilization. Generally, dispersions are prepared by incorporating an active agent into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. In many cases, it will be preferable to include isotonic agents.

Various methods of delivery can be used to deliver the test agent and will in part be dependent upon the agent and its bioavailability. For example, small molecules or other agents that are bioavailable may be administered orally, whereas protein-based agents are generally but not exclusively administered parenterally. Certain agents may be administered systemically, while others may be more beneficial with a local delivery. The method of delivery will be apparent to one skilled in the art upon reading the specification and can be determined in view of the specific properties of the test agent.

It is understood that the effective amount of a test agent may vary depending on the nature of the effect desired, frequency of treatment, any concurrent treatment, the health, weight of the recipient, and the like. See, e.g., Berkow et al., eds., Merck Manual, 16th edition, Merck and Co., Rahway, N.J. (1992); Goodman et al., eds., Goodman and Gilman's The Pharmacological Basis of Therapeutics, 8th edition, Pergamon Press, Inc., Elmsford, N. Y. (1990); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston (1985), Katzung, Basic and Clinical Pharmacology, Appleton and Lange, Norwalk, Conn. (1992), which references and references cited therein, are entirely incorporated herein by reference.

### Definitions

For the purposes of clarity and a concise description, features can be described herein as part of the same or separate embodiments; however, it will be appreciated that the scope of the invention may include embodiments having combinations of all or some of the features described.

The terminology used herein is for the purpose of describing particular embodiments only and is not intended to be limiting of the invention. Unless defined otherwise, all technical and scientific terms used herein have the same meaning as commonly understood by one of ordinary skill in the art to which this invention belongs. The following definitions are intended to aid the reader in understanding the present invention but are not intended to vary or otherwise limit the meaning of such terms unless specifically indicated.

As used herein, the indefinite articles “a”, “an” and “the” should be understood to include plural reference unless the context clearly indicates otherwise. Thus, for example, reference to “an inhibitor” refers to one or more agents with the ability to inhibit a target molecule, and reference to “the method” includes reference to equivalent steps and methods known to those skilled in the art, and so forth.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the term “about” means plus or minus 10% of the indicated value. For example, about 100 means from 90 to 110.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

The term “small molecule” refers to a molecule of a size comparable to those organic molecules generally used in pharmaceuticals. The term excludes biological macromolecules (e.g., proteins, nucleic acids, etc.). Small organic molecules include those that range in size up to about 5000 Da, including up to 2000 Da, and including up to about 1000 Da.

A “test agent” as used herein refers to any agent that is a candidate to treat a disease or symptom thereof. Such agents include, but are not limited to, peptides; proteins (including derivatized or labeled proteins); antibodies or fragments thereof; small molecules; aptamers; carbohydrates and/or other non-protein binding moieties; derivatives and fragments of naturally occurring binding partners; peptidomimetics; and pharmacophores.

The term “pharmacophore” is used herein in an unconventional manner. Although the term conventionally means a geometric and/or chemical description of a class or collection of compounds, as used here the term means a compound that has a specific biochemical activity or binding property conferred by the 3-dimensional physical shape of the compound and the electrochemical properties of the atoms making up the compound. Thus, as used here the term “pharmacophore” is a compound and not a description of a collection of compounds which have defined characteristics. Specifically, a “pharmacophore” is a compound with those characteristics.

### EXAMPLES

The following examples are put forth so as to provide those of ordinary skill in the art with a complete disclosure and description of how to make and use the present invention and is not intended to limit the scope of what the inventors regard as their invention, nor is the example intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Assay to Discover Compounds to Promote Remyelination

### INTRODUCTION

A barrier to therapeutic advances in multiple sclerosis (MS) is the inability to overcome the inhibitory lesion environment which contributes to failed remyelination and axonal loss in the progressive stages of disease (1). The blood protein fibrinogen is deposited in the CNS after vascular damage and is a component of the inhibitory lesion environment (1). Fibrinogen promotes CNS inflammation, demyelination, and axonal damage, and blocks oligodendrocyte progenitor cells (OPCs) from differentiating into mature myelinating oligodendrocytes (OLs) (2-5). In turn, perivascular OPC clusters can contribute to persistent blood-brain barrier (BBB) disruption and accumulation of fibrinogen in chronic MS lesions. (6).

Discovery of drugs that overcome the fibrinogen-rich, inhibitory MS lesion environment would provide a desperately needed alternative therapeutic avenue for MS. Although therapeutic depletion of fibrinogen can enhance remyelination in demyelinating animal models, the potential for hemorrhagic complications may limit its clinical use (2). Compounds that promote OPC differentiation have recently been identified in unbiased drug screens; however, it is not known whether these myelin-promoting compounds can overcome fibrinogen's inhibitory effects on myelin repair in inflammatory demyelination (7-10).

Here, the development of a novel, medium-throughput fibrinogen OPC-inhibition Assay is described. It is shown that none of the recently discovered myelin-promoting compounds are able to rescue fibrinogen's effects on OPCs in this assay. Only the BMP Type I receptor inhibitors restore OPC differentiation to mature OLs in the presence of fibrinogen. The data shows that known myelin-promoting compounds may not be able to overcome extrinsic inhibitors like fibrinogen in the MS lesion environment and that targeting fibrinogen-induced BMP signaling may be an alternative therapeutic avenue to promote functional recovery in MS. The novel assay described herein can be used to screen compounds that overcome the fibrinogen-driven inhibition of OPC maturation and remyelination.

### Materials and Methods/Results/Discussion

### A. Development of a Medium-Throughput Fibrinogen-OPC Inhibition Assay

In order to miniaturize the assay to a 96-well plate format we first optimized OPC density, fibrinogen concentration, staining procedures as well as the protocol used for image analysis (FIG. 1′A). For the optimized protocol primary rat O4+ OPCs are isolated by immunopanning. ˜5×10⁵ cells per 10 cm dish were plated and incubated for 3 days to allow for OPC proliferation. Cells were then passaged using Accutase and re-plated into 96 well plates at 5×10³ cells/well and allowed to recover for 1 day prior to experiments. To achieve an optimal difference between control and fibrinogen condition, fibrinogen needed to be added at a concentration of 2.5 mg/mL at a minimum. OPCs were allowed to differentiate for 3 days before fixation and subsequent staining procedure. OPCs were stained with Hoechst dye (nuclei), and antibodies to MBP (oligodendrocytes) and GFAP (astrocytes). Images were acquired using the Arrayscan XTI instrument. To reduce well-to-well variability 25 images were taken with a 10×objective, covering the about 80% of well surface area. Images were analyzed using the HCS Studio software. Total cell count was calculated based on the number of Hoechst+ nuclei. To quantify the percentage of total cells positive for either MBP or GFAP, a ring was expanded out from the nuclear mask (Hoechst dye) to include the cell body. For MBP we extended the ring large enough around the nucleus Nuclei with green fluorescence cell body (MBP+488 nm) were calculated as percentage of MBP+ cells of total cell count and nuclei with red cell body (GFAP+, 549 or 647 nm) as percentage of GFAP+ cells.

### B. Development of a High-Content Fibrinogen-OPC Inhibition Assay in 384-Well Format

We used the established assay parameters from our 96-well assay to develop an automatable, 384-well high content screening assay. OPCs were obtained and cultured as described above. To adapt the assay to 384-well format different cell densities per well were tested. 1×10³ cells/well was determined to be the optimal cell number. Cells were plated manually with a multichannel pipette. The following steps were performed using the Agilent BRAVO liquid handler: After 24 hours of culture, proliferation medium was taken out of each well, DPBS containing glucose and sodium pyruvate was added for a brief wash to ensure that all proliferation factors were removed. After removing the DPBS solution, 50 μL of medium containing a 2-fold concentration of differentiation factors+ compounds were added from a pre-dilution polypropylene plate. After one hour incubation 50 μL of regular medium containing a 2-fold concentration of fibrinogen were added manually with a multichannel pipette, keeping the solution on a heating platform. After 3 days of incubation the cells are stained according to the same protocol as described for the 96-well plate format but using a BioTek EL406 liquid handler for all steps of the protocol. Image acquisition and analysis followed as described above using the Thermo Scientific Arrayscan XTI.

### DISCUSSION

Scaling up number of OPCs available per isolation which increased the yield from 1.5 million cells per prep to 5 million cell per prep after proliferation. Proliferation can be carried out for 3-4 days in 10 cm plates in PDGF-AA and NT3 containing OPC media. ACCUTASE™ can be used to aid in passaging cells for about 5-minute incubation at 37°. Re-plating can be done into 96-well or 384-well plates, for example, in a 96-well plate=5000 cells per well; 384-well plate=1000 cells per well, for a time in differentiation media (e.g., about 3 days) for reproducible OPC differentiation. Fibrinogen can be used at concentration of at least about 2.5 mg/ml to inhibit OPC differentiation to MBP+ cells and maximize OPC differentiation to GFAP+ cells.

Workflow was developed to test many compounds at the same time in the same plate. For example, multichannel administration of 2× compounds followed by 2× fibrinogen can be used to decrease time needed to administer compounds and fibrinogen outside of the incubator. Programming and use of Agilent BRAVO liquid handler for 384-well format can be used to minimize dislodgment of cells during media transfer. BioTek EL406 liquid handler can be used for immunostaining procedure in 384-well format.

Cells/cellular components/proteins of interest can be labeled/stained, for example, MBP (1:250) and GFAP (1:500) primary antibodies, secondary fluorescent antibodies (1:500), and Hoechst nuclear dye (2 μg/mL) and used for for automated detection. Automated image acquisition can be carried out with the Thermo Scientific Arrayscan XTI, for example, acquisition of 25 images at 10× to maximize the quantified area of each well (˜80% coverage of well surface area of a 96-well plate well). A large area is need as cells can tend to cluster in some areas, so full-well imaging more accurately captures the treatment effect and reduces well-to-well variability in quantification. The stains and labels, for example, MBP+ and GFAP+ cell, can undergo automated quantification (quantification methods can be designed using the HCS Studio software (Thermo Scientific)). As an example, to quantify the percentage of total cells positive for GFAP, a ring was expanded out from the nuclear mask (Hoechst dye) to include the cell body and for MBP+ cells, the ring was extended beyond the cell body to include cell processes, ensuring that only mature OLs are included in the analysis. A cell was determined as positive by the software if the fluorescence intensity measured within the ring was above the threshold set for fluorescence intensity produced in secondary antibody only controls. Overall, this technique eliminates bias and reduces well-to-well and plate-to-plate variability in quantification.

Further, all of the steps described herein increase the speed in which results are obtained. Manual image acquisition and quantification of 96-well plates takes 1-2 weeks whereas the automated image acquisition and quantification can be done in 1 day.

### BIBLIOGRAPHY

1. Petersen, M. A., Ryu, J. K. & Akassoglou, K. Fibrinogen in neurological diseases: mechanisms, imaging and therapeutics. Nat Rev Neurosci 19, 283-301 (2018).
2. Petersen, M. A. et al. Fibrinogen Activates BMP Signaling in Oligodendrocyte Progenitor Cells and Inhibits Remyelination after Vascular Damage. Neuron 96, 1003-1012 e7 (2017).
3. Davalos, D. et al. Fibrinogen-induced perivascular microglial clustering is required for the development of axonal damage in neuroinflammation. Nat Commun 3, 1227 (2012).
4. Ryu, J. K. et al. Blood coagulation protein fibrinogen promotes autoimmunity and demyelination via chemokine release and antigen presentation. Nat Commun 6, 8164 (2015).
5. Merlini, M. et al. Fibrinogen Induces Microglia-Mediated Spine Elimination and Cognitive Impairment in an Alzheimer's Disease Model. Neuron 101, 1099-1108 e6 (2019).
6. Niu, J. et al. Aberrant oligodendroglial-vascular interactions disrupt the blood-brain barrier, triggering CNS inflammation. Nat Neurosci 22, 709-718 (2019).
7. Mei, F. et al. Identification of the Kappa-Opioid Receptor as a Therapeutic Target for Oligodendrocyte Remyelination. J Neurosci 36, 7925-35 (2016).
8. Mei, F. et al. Micropillar arrays as a high-throughput screening platform for therapeutics in multiple sclerosis. Nat Med 20, 954-960 (2014).
9. Najm, F. J. et al. Drug-based modulation of endogenous stem cells promotes functional remyelination in vivo. Nature 522, 216-20 (2015).
10. Fancy, S. P. et al. Axin2 as regulatory and therapeutic target in newborn brain injury and remyelination. Nat Neurosci 14, 1009-16 (2011).
11. Green, A. J. et al. Clemastine fumarate as a remyelinating therapy for multiple sclerosis (ReBUILD): a randomised, controlled, double-blind, crossover trial. Lancet 390, 2481-2489 (2017).
12. Mohedas, A. H. et al. Development of an ALK2-biased BMP type I receptor kinase inhibitor. ACS Chem Biol 8, 1291-302 (2013).
13. Adams, R. A. et al. The fibrin-derived gamma377-395 peptide inhibits microglia activation and suppresses relapsing paralysis in central nervous system autoimmune disease. J Exp Med 204, 571-82 (2007).
14. Ryu, J. K. et al. Fibrin-targeting immunotherapy protects against neuroinflammation and neurodegeneration. Nat Immunol 19, 1212-1223 (2018).
Forbes T A, Gallo V. All wrapped up: environmental effects on myelination. Trends Neurosci 2017; 40(9): 572-87.
Hammond T R, Gadea A, Dupree J, Kerninon C, Nait-Oumesmar B, Aguirre A, et al. Astrocyte-derived endothelin-1 inhibits remyelination through notch activation. Neuron 2014; 81(3): 588-602.
Karamita M, Barnum C, Mobius W, Tansey M G, Szymkowski D E, Lassmann H, et al. Therapeutic inhibition of soluble brain TNF promotes remyelination by increasing myelin phagocytosis by microglia. JCI Insight 2017; 2(8).
Keough M B, Rogers J A, Zhang P, Jensen S K, Stephenson E L, Chen T, et al. An inhibitor of chondroitin sulfate proteoglycan synthesis promotes central nervous system remyelination. Nat Commun 2016; 7: 11312.
Kirby L, Jin J, Cardona J G, Smith M D, Martin K A, Wang J, et al. Oligodendrocyte precursor cells present antigen and are cytotoxic targets in inflammatory demyelination. Nat Commun 2019; 10(1): 3887.
Kotter M R, Li W W, Zhao C, Franklin R J. Myelin impairs CNS remyelination by inhibiting oligodendrocyte precursor cell differentiation. J Neurosci 2006; 26(1): 328-32.
Mabie P C, Mehler M F, Marmur R, Papavasiliou A, Song Q, Kessler J A. Bone morphogenetic proteins induce astroglial differentiation of oligodendroglial-astroglial progenitor cells. J Neurosci 1997; 17(11): 4112-20.
Petersen M A, Ryu J K, Akassoglou K. Fibrinogen in neurological diseases: mechanisms, imaging and therapeutics. Nat Rev Neurosci 2018; 19(5): 283-301.
Srivastava T, Diba P, Dean J M, Banine F, Shaver D, Hagen M, et al. A TLR/AKT/FoxO3 immune tolerance-like pathway disrupts the repair capacity of oligodendrocyte progenitors. J Clin Invest 2018; 128(5): 2025-41.
Stoffels J M, de Jonge J C, Stancic M, Nomden A, van Strien M E, Ma D, et al. Fibronectin aggregation in multiple sclerosis lesions impairs remyelination. Brain 2013; 136(Pt 1): 116-31.
Syed Y A, Hand E, Mobius W, Zhao C, Hofer M, Nave K A, et al. Inhibition of CNS remyelination by the presence of semaphorin 3A. J Neurosci 2011; 31(10): 3719-28.

### Example 2

### BMP Receptor Blockade Overcomes Extrinsic Inhibition of Remyelination and Restores Neurovascular Homeostasis

### INTRODUCTION

Regeneration of CNS myelin fails in several neurological diseases, such as multiple sclerosis, neonatal brain injury, and stroke (Franklin and Ffrench-Constant, 2017). In these conditions, cell-extrinsic cues in the microenvironment inhibit remyelination by blocking multipotent OPCs from differentiating into mature, myelin-producing oligodendrocytes (OLs) (Forbes and Gallo, 2017). A critical barrier to therapeutic advances in chronic demyelinating diseases like multiple sclerosis is the inability to overcome this inhibitory lesion environment and halt disease progression (Reich et al., 2018). Small molecules that enhance intrinsic pathways of OPC differentiation and remyelination have been identified in drug screens (Fancy et al., 2011; Deshmukh et al., 2013; Mei et al., 2014; Najm et al., 2015; Mei et al., 2016). However, these drugs have failed to overcome disease-relevant extrinsic inhibitors of OPC differentiation such as chondroitin sulfate proteoglycans (CSPGs) and inflammatory cytokines and fail to promote OL differentiation in aged OPCs or OPCs from multiple sclerosis patients in an inflammatory environment (Keough et al., 2016; Neumann et al., 2019; Starost et al., 2020). Whether promyelinating compounds can overcome the inhibitory microenvironment at sites of increased vascular permeability remains unknown.

In multiple sclerosis, blood-brain barrier (BBB) disruption allows the blood coagulation factor fibrinogen to enter the CNS (Petersen et al., 2018). Fibrinogen deposition is one of the earliest events in multiple sclerosis pathogenesis and persists in chronically demyelinated lesions but is minimal in remyelinated lesions and absent in normal white matter (Vos et al., 2005; Petersen et al., 2017; Lee et al., 2018). In progressive multiple sclerosis, fibrinogen is detected in the cortex and cerebrospinal fluid and correlates with neuronal and cortical loss (Yates et al., 2017; Magliozzi et al., 2019). In demyelinating injury models, genetic or pharmacologic depletion of fibrinogen promotes remyelination in the CNS and peripheral nervous system (Akassoglou et al., 2002; Petersen et al., 2017). Fibrinogen activates BMP receptor signaling in OPCs and neural precursor cells to inhibit remyelination and neurogenesis, respectively (Petersen et al., 2017; Pous et al., 2020). Fibrinogen induces a cell fate switch of NG2+(encoded by CSPG-4) OPCs to astrocytes via BMP receptor activation (Petersen et al., 2017), suggesting a role for fibrinogen in extrinsic inhibition of remyelination by inducing OPC-derived astrogenesis in the neurovascular niche. Furthermore, when fibrinogen is converted to fibrin, it induces oxidative stress and pro inflammatory polarization of microglia and macrophages (Ryu et al., 2015; Mendiola et al., 2020), which is toxic to OPCs and contributes to remyelination failure (Back et al., 1998; Miron et al., 2013). This suggests a role for increased vascular permeability and fibrinogen deposition in the maintenance of an inhibitory microenvironment in chronic neurological diseases. However, the remodeling of the neurovascular niche at sites of BBB disruption and its relationship with remyelination failure remains poorly understood.

Here, it is shown that extrinsic inhibition of remyelination by fibrinogen activates signaling pathways in OPCs that could not be overcome by known promyelinating compounds, such as clemastine. In contrast, inhibition of BMP signaling rescued the inhibitory effects of fibrinogen on remyelination by restoring the cell fate of OPCs to mature OLs with therapeutic effects in chronic EAE models. By integrating transcriptomics with in vivo two-photon (2P) imaging co-registered with electron microscopy in chronic neuroinflammatory lesions, it is shown that OPCs accumulate at sites of fibrinogen deposition with active BMP signaling and limited remyelination. Thus, known promyelinating compounds do not overcome BMP receptor activation and abortive OPC differentiation by fibrinogen, suggesting that BMP pathway inhibition may enhance the regenerative potential of the promyelinating progenitor niche at sites of cerebrovascular damage.

### Materials and Methods

### Animals

C57BL/6, NOD, B6.Cg-Tg(Cspg4-cre/Esr1*)BAkik/J (NG2-CreER^{TM}),¹ B6.Cg-Gt(ROSA)26^{Sortm14(CAG-tdTomato)Hze}/J (Rosa^{tdTomato}),² and B6.129P-Cx3cr1^{tm1Litt}/J (CX3CR1^{GFP})³ mice were purchased from the Jackson Laboratory. Mice were housed in groups of five per cage under standard vivarium conditions and a 12-h light/dark cycle. Sprague-Dawley female rats with litters were purchased from Charles River, and P1-P7 male and female rats were used for OPC isolations. All animal protocols were approved by the Committee of Animal Research at the University of California, San Francisco, and in accordance with the National Institutes of Health and ARRIVE guidelines.

### EAE Induction and Clinical Scoring

Active EAE was induced in 9- to 10-week-old NG2-CreER^{TM}:Rosa^{tdTomato/+}:Cx3cr1^{GFP/+} female mice 35-40 days after the last tamoxifen injection by subcutaneous immunization with 75 μg MOG₃₅₋₅₅ peptide (MEVGWYRSPFSRVVHLYRNGK (SEQ ID NO: 1); Auspep), in incomplete Freund's Adjuvant (Sigma-Aldrich) supplemented with 400 μg of heat-inactivated Mycobacterium tuberculosis H37Ra (Difco Laboratories). At day 0 and 2 after immunization, mice were given intraperitoneal injection of 200 ng pertussis toxin (Sigma-Aldrich). For the chronic NOD EAE model, 10- to 12-week-old NOD mice were immunized with 150 μg MOG₃₅₋₅₅ peptide, followed by administration of 200 ng pertussis toxin on days 0 and 2 as described.⁴

For therapeutic treatment, at peak+2d mice were administered 6 mg/kg LDN-212854 (Axon Medchem #2201) or saline twice daily (10-14 hrs apart) for 14 days. Mice were randomly assigned to treatment groups, scored and drug-treated in a blinded manner. Experimental groups were unblinded to treatment assignment at the end of the experiments to ensure experimenter bias was not introduced. Mice that did not develop symptoms of EAE were excluded from treatment and analysis. Mice were weighed and scored daily. Neurological deficits were assessed on a five-point scale by observers blinded to treatment: 0, no symptoms; 1, loss of tail tone; 2, ataxia; 3, hindlimb paralysis; 4 hindlimb and forelimb paralysis; 5, moribund. EAE peak was defined by score >2.5.

### Fluorescence-Activated Cell Sorting of NG2 Cells

For sorting NG2 cells, spinal cord tissues were collected from perfused female mice as previously described.⁵ Single-cell suspensions were prepared from entire spinal cords following the adult brain dissociation (ABD) kit manufacturer's instructions with modification (Miltenyi Biotec). Briefly, minced tissues were individually incubated with ABD Mix 1 containing 15 μM actinomycin D (ActD; Sigma)⁶ for 15 min at 34° C., and then ABD Mix 2 was added to the solution for 10 min at 34° C. Tissues were gently triturated and then incubated for 10 min at 34° C. Homogenized tissue solutions were passed through 70-μm smartstrainer (Miltenyi Biotec), washed with cold Dulbecco's phosphate-buffered saline and centrifuged at 450×g for 7 min at 4° C. Tissue debris was removed following the ABD Kit debris removal step instructions and then passed through 30-μm smartstrainer (Miltenyi Biotec) and centrifuged at 450×g for 7 min at 4° C. All steps above were performed in the presence of 3 μM ActD. Single-cell suspensions were incubated with 1 μM Sytox blue live/dead stain (Thermo Fisher Scientific) for 5 min at 4° C. and then cell sorting was performed on an FACSARIA II (BD Biosciences) with BD FACSDiva™ v8 software. All cells were gated based on SSC-A and FSC-A size and then doublet discrimination was performed by FSC-H and FSC-W parameters. Sytox blue⁻ NG2^{tdtomato+} cells were sorted directly into tubes containing RLT plus lysis buffer (Qiagen) supplemented with 1%2-mercaptoenthanol (Sigma) and 0.25% reagent DX (Qiagen). Cell lysates were frozen on dry ice and immediately stored at −80° C. until use. For determination of TFPI and MHC class II expression, single cell suspension of C57BL/6 spinal cord tissues were prepared as above without adding ActD. Cells were incubated with Fc Block (BioLegend) for 15 min on ice followed by fluorescently conjugated Abs and anti-TFPI in FACS staining buffers (BD) for 30 min on ice. Cells were then stained with aqua live/dead staining kit (Thermo Fisher Scientific) along with fluorescently conjugated secondary antibody in PBS on ice for 30 min. Samples were run on the LSRFortessa (BD Biosciences) immediately with BD FACSDiva™ v8 software. All FACS plots were generated with Flowjo. Following antibodies were used: APC/Cy7 anti-mouse CD11b (BioLegend, #101225, 1:200), PE anti-mouse CD3 (BioLegend, #100206, 1:200), PE/Cy7 anti-mouse PDGFRA (Invitrogen, #25-1401-82, 1:50), Alexa Fluor 488 anti-mouse PDGFRB (Invitrogen, 53-1402-82, 1:50), BV650 anti-mouse MHCII (BD, #743873, 1:200), rabbit anti-mouse TFPI (Invitrogen, PA5-34578, 1:100), BV421 Donkey anti-rabbit IgG (Biolegend, 406410, 1:200) and LIVE/DEAD™ fixable aqua dead cell stain kit (Invitrogen, L34957, 1:500).

### Bulk RNA Sequencing

Frozen NG2 cell lysates in RLT buffer were thawed at 24° C. and then lysed using the QIAshredder (Qiagen) following manufacturer's instructions. Total RNA was isolated from cell lysates using the RNAeasy micro kit without modification (Qiagen). RNA quality and quantity were determined by Bioanalyzer pico chip analysis (Agilent) and all samples with RNA integrity number >8 were used for RNA-seq library preparation. cDNA libraries were generated from total RNA using the Ovation RNA-seq System V2 (NuGEN). Libraries were quantified and quality checked by KAPA qPCR (Roche) and Bioanalyzer DNA chip analysis (Agilent), respectively. Libraries were pooled and paired-end 75 base pair read length sequenced across 8 lanes on a Nextseq500 (Illumnia), for a sequencing depth of >40 million reads per library. FASTQ files were generated in Biospace following manufacturer's guidelines (Illumina).

### Analysis of Bulk RNA Sequencing

For each sample, read 1 and read 2 FASTQ files were separately catenated, and Illumnia adaptors were trimmed and FASTQ files were quality checked using FASTQC. Next, sequencing reads were aligned to mouse reference genome mm10 with STAR and then counts per gene were quantified by featureCounts as previously described.⁵ DEGs were identified by EdgeR (version 3.24.3),⁷ using cutoffs of log 2 fold change of >1 or <−1 and false discovery rate (FDR) p-value<0.05. Using R (version 3.5.0), K-means HOPACH (version 2.42.0) clustering analysis of DEGs was visualized using pheatmap package (version 1.0.12) and volcano plots were generated with ggplot2 package (version 3.2.1).

### Functional Enrichment and Gene Network Analysis

Functional enrichment analysis of DEGs clustered by HOPACH was performed in Metascape using default parameters,⁸ and significant gene ontology (GO) terms were identified by FDR p-value<0.05. Using RNA-seq normalized counts per million dataset, gene network analyses were performed with GSEA with molecular signatures database biological process for GO (C5.bp.v7.1symbols.gmt) using default settings.^{9, 10} GO terms with p-value<0.10 were used for Enrichment Map Visualization using Cytoscape (version 3.7.2)¹¹ and were unbiasedly clustered using the plugin AutoAnnotate (version 1.3.2) with default settings.

### In Vivo Multiphoton Microscopy

An Ultima IV 2P microscope (Prairie Technologies/Bruker) equipped with a Mai Tai eHP DeepSee and an Insight X3 Ti:sapphire femtosecond laser (pulse width<120 fs, tuning range 690-1040 nm (Mai Tai) and 680-1300 nm (Insight X3), repetition rate 80 MHz; Spectra-Physics/Newport) was used. The lasers were tuned to an excitation wavelength of 910-1150 nm depending on the fluorophore(s). Imaging was performed ˜80-120 μm below the dura mater using an Olympus 25×1.05 NA with 1.6 zoom or a Nikon 10×0.4 NA water-immersion lenses with either a 1.0-1.5-μm or a 3-4-μm z-step, for 40× or 10× magnification respectively. The maximum laser power exiting the objective was <40 mW during all imaging experiments. An IR-blocking filter and 560-nm dichroic were placed in the primary emission beam path before the non-descanned detectors. A 660-nm dichroic and a 692/24-nm+607/45-nm bandpass filter were used to separate Mito Tracker Red/far red and tdTomato/rhodamine fluorescence emission, respectively; a 520-nm dichroic and a 542/27-nm+494/41-nm bandpass filter were used to separate YFP and GFP fluorescence emission, respectively.

### In Vivo Spinal Cord Imaging

In vivo spinal cord imaging was performed as previously described.¹² Briefly, the spinal cord was exposed at the desired level (T11) through a single laminectomy, and mice were positioned on a spinal stabilization device. Flow-It® ALC (Pentron) was used to create a well around the exposed spinal cord and a drop of pre-warmed (37° C.) artificial cerebrospinal fluid (ACSF, HEPES-based; in mM: 125 NaCl, 10 glucose, 10 HEPES, 3.1 CaCl2, 2.7 KCl, and 1.3 MgCl2; pH 7.4) was applied, preceded by gentle flushing of the dura mater with pre-warmed ACSF to clean and remove potential dural bleedings. Mice were excluded from the study if they sustained accidental injury during the laminectomy or there were signs of (sub-) dural hemorrhage, as these events would cause inflammatory and other neurodegenerative responses unrelated to the experimental design. A 100-μl solution of 3%70-kDa Oregon green-conjugated dextran (Thermo Fisher Scientific) in ACSF was injected retro-orbitally to label the vasculature, after which the mouse was placed underneath the 2P imaging microscope. For in vivo myelin imaging, the meninges (dura mater and arachnoidea) were carefully removed using a hypodermic needle and the underlying exposed spinal cord was bathed with MitoTracker Deep Red (Thermo Fisher Scientific) dissolved in ACSF at a concentration of 8 μM for 30 min.¹³ The spinal cord was then carefully washed 4-5 times with pre-warmed ACSF before the imaging session.

### Processing of In Vivo Imaging Data

To generate images for figures, z-stacks were intensity-projected along the z-axis using the ImageJ (NIH) summation projection algorithm to recreate two-dimensional representations of the imaged volumes. Images were adjusted for brightness/contrast, background noise and sharpness with ImageJ using Subtract Background, Remove Outliers and Unsharp mask algorithms. The spectral unmixing algorithm in ImageJ was used to separate the GFP and YFP signals, which were subsequently pseudocolored.

### Quantification of Cell Clusters

Z-stacks of images from NG2-CreER^{TM}:Rosa^{tdTomato/+}:Cx3cr1_{GFP/+} healthy control or EAE-challenged mice were z-projected and automatically thresholded (default algorithm of ImageJ), to account for signal intensity differences between experiments. NG2 and microglial clusters were defined as areas where 4 or more cell bodies were touching each other, and cell density was at least two-fold higher than in healthy appearing spinal cord. Cluster number and distance to the closest blood vessel were measured with ImageJ.

### Myelin Circularity

Myelin damage was quantified with myelin circularity. A value of 1.0 indicates a perfect circle (as seen in degenerating myelin in longitudinal sections); as the value approaches 0.0, it indicates an increasingly noncircular, linear shape (longitudinal section of normal myelinated fiber).

### Electron Microscopy

Tissue Preparation for SBEM. In vivo 2P imaging of NG2-CreER^{TM}:Rosa^{tdTomato/+}:Cx3cr1^{GFP/+} mice was performed at chronic EAE to reveal tdTomato+NG2-lineage cells, microglia, and the vasculature visualized with dextran. After the imaging session, the animal was perfused with Ringers solution followed by 0.5% glutaraldehyde/2% PFA in cacodylate. The region of spinal cord under the imaging window was cut from the perfused cord and post-fixed for 2 hours in cold 0.5% glutaraldehyde/2% PFA in cacodylate. The specimen was then post-fixed overnight in cold 4% PFA in cacodylate. The dorsal aspect of the cord was cut into 150 μm thick horizontal vibratome sections. The sections were post-fixed overnight in cold 2% glutaraldehyde in cacodylate. The sections were stained as previously described.¹⁴ Briefly, the tissue was stained with 2% osmium tetroxide (Ted Pella) in 0.15M cacodylate, 0.5% aq. thiocarbohydrazide (Electron Microscopy Sciences), 2% aq. osmium tetroxide, 2% aq. uranyl acetate (Ted Pella), and lead aspartate,¹⁵ with thorough washing with water between each staining solution. The sections were then dehydrated through ethanol and acetone and then infiltrated with Durcupan ACM (Millipore Sigma). The sections were flat-embedded between glass slides coated with mold-release compound (Electron Microscopy Sciences, Hatfield PA) and cured at 60° C. for 72 hours.

X-ray Microscopy and ROI Targeting for SBEM. Specimens were imaged with XRM in order to find and orient ROIs for SBEM imaging.¹⁶ Specimens were scanned with a Zeiss Versa 510. Initial scans of whole vibratome slices were collected with a 0.4×objective at 80 kV and a pixel size of approximately 5 μm. After comparison of the vasculature observed in the XRM and two-photon volumes, the ROI was identified and cut out using a razor blade. The specimens were glued onto a piece of ACLAR (Ted Pella), itself glued to a dummy block, using cyanoacrylate glue, with the ventral aspect of the vibratome slice facing up. Using the XRM volume as guidance, the specimen was approached with a glass blade on a Leica EM UC6 ultramicrotome so that the cutting plane was parallel with the desired final cutting plane in the SBEM. Once excess epoxy was removed and tissue exposed, the specimen was removed from the dummy block and attached to an A3 SBEM specimen pin (RMC Boeckeler) using conductive silver epoxy (Ted Pella), this time with the dorsal aspect facing up. The A3 pin was placed in the A3 specimen holder and scanned with XRM using the 4× objective at 80 kV for a pixel size of approximately 1.5 μm. This XRM volume was used to precisely adjust the tilt of the specimen block, remove excess resin from the dorsal aspect of the block, and identify the ROI location for SBEM imaging.

SBEM Imaging. Specimens were imaged on a Zeiss Gemini 300 VP SEM equipped with a focal charge compensation system and a Gatan 2XP 3 View system. Volumes were collected at 2.5 kV with 1 usec dwell time, 10 nm pixels, 50 nm step size, and focal gas injection with nitrogen gas turned on. The scope was run in analytic mode and high current mode. The resulting stacks of images were aligned using a custom Python script using IMOD programs.¹⁷

### OPC-X-Screen

Primary rat O4⁺ OPCs were isolated as previously described by immunopanning papain-dissociated cortical cell suspensions sequentially on three dishes: RAN-2 (negative selection), O1 (negative selection), and O4 (positive selection).¹⁸ O4+ OPCs were seeded on polyethyleneimine (PEI, Sigma-Aldrich)-coated 10 cm culture plate at an initial density of 5×10⁵ cells per plate and expanded in proliferation media for 3 days in a 5% CO2 incubator at 37° C. Cells were then passaged using Accutase and re-plated into PEI-coated μClear® 96 well plates (Greiner Bio-One) at 5×10³ cells per well. Cells were incubated in proliferation media for 1 day prior to experimental treatments which were performed in differentiation media. The chemically defined base media was DMEM (4.5 g/L glucose, +pyruvate, +glutamine; Thermo Fisher Scientific), 1× B27 (Thermo Fisher Scientific), 1× N2 (Thermo Fisher Scientific), 1% penicillin-streptomycin (Thermo Fisher Scientific), and 50 ng ml⁻¹ NT3 (Peprotech). Proliferation media consisted of the base media supplemented with 20 ng ml⁻¹ PDGF-AA (Peprotech). Differentiation media consisted of the base media supplemented with 20 ng ml⁻¹ CNTF (Peprotech) and 40 ng ml⁻¹ triiodothyronine (T3, Sigma-Aldrich) with no PDGF-AA. “Slow” differentiation media (base media with no NT3 or additional growth factors and no T3) was used in clemastine dose-response studies to recapitulate the conditions in previous reports.¹⁹

To mimic the inhibitory lesion environment, fibrinogen (Millipore Sigma) was added to differentiation media at a concentration of 1.5 mg ml⁻¹ for the myelin-promoting compound screen and 2.5 mg ml⁻¹ for all other in vitro studies, which are physiologic plasma concentrations known to inhibit OPC differentiation to mature OLs.¹⁸ Myelin-promoting compounds were dissolved in DMSO and added to quadruplicate wells at a concentration previously shown to promote OPC differentiation to OLs 1 hour before fibrinogen treatment. Final compound concentrations were: benztropine 1 μM,¹⁹ clemastine 1 μM,¹⁹ quetiapine 1 μM,¹⁹ miconazole 1 μM,²⁰ clobetasol 5 μM,²⁰ (±)U-50488 1 μM,²¹ and XAV-939 0.1 μM²². DMH1 (1 μM)¹⁸ served as a positive control in all assay plates. Cells were exposed to a maximum DMSO concentration of 0.1%, and controls contained an equal concentration of DMSO. All conditions were tested in quadruplicate wells and repeated in three independent experiments for an N=3 biological replicates. For dose response curves, LDN-212854 and clemastine were added to quadruplicate wells in three-fold serial dilutions (5 μM to 2 nM) 1 hour prior to fibrinogen treatment. Dose-response experiments were repeated in two or three independent experiments. Cells were allowed to differentiate for 3 days prior to fixation, staining, and quantification. For testing the combination of a BMP receptor inhibitor and another promyelinating compound, LDN-212854 (0.1 μM) and clemastine (0.5 μM) were added alone or together in quadruplicate wells 1 hour before fibrinogen treatment in three independent experiments. Cells were allowed to differentiate for 2 days prior to fixation, staining, and quantification.

OPCs were fixed with 4% paraformaldehyde, blocked and permeabilized in 5% normal goat serum/0.1% Triton-X100, and stained with 2 μg/mL Hoechst nuclear dye (Thermo Fisher Scientific), anti-MBP antibody (Abcam ab92406 or Abcam ab7349), and anti-GFAP antibody (Cell Signaling #12389) followed by goat secondary antibodies (Thermo Scientific). Images were acquired with the Arrayscan XTI instrument (Thermo Scientific) using a 10× objective, a 386/23 filter for detection of Hoechst dye, a 485/20 filter to detect MBP/Alexafluor-488 and a 549/18 filter to detect GFAP/Alexafluor-546 fluorescence. To reduce well-to-well variability, 25 images were taken covering approximately 80% of the well surface area. Images were analyzed using the HCS Studio software (Thermo Scientific). Total cell count was calculated based on the number of Hoechst⁺ nuclei. To quantify the percentage of total cells positive for either MBP or GFAP, a ring was expanded out from the nuclear mask (Hoechst dye) to include the cell body (GFAP⁺ cells). For MBP+ cells the ring was extended beyond the cell body to include OLs processes, ensuring that only mature OLs would be included in the analysis. Using the HCS Studio software the percentage of MBP⁺ and GFAP⁺ cells was calculated based on the number of MBP⁺ and GFAP⁺ cells per total number of cells. A cell was determined as positive by the software if the fluorescence intensity measured within the ring was above the threshold set for fluorescence intensity produced in secondary antibody only controls.

### Immunohistochemistry

Mice were transcardially perfused with 4% PFA under deep avertin or ketamine/xylazine anesthesia. Tissue was removed, post-fixed overnight in 4% PFA, cryoprotected in 30% sucrose/PBS, frozen in Neg-50 media (Thermo Scientific Scientific), cryosectioned into 10-12 μm sections, and placed on Tissue Tack microscope slides (Polysciences, Inc). Sections were permeabilized in 0.1-0.3% Triton X-100, blocked with 5% BSA or 5% normal donkey serum, and incubated with primary antibodies overnight at 4° C. and then fluorescent secondary antibodies for 1-2 h at room temperature. Slides were coverslipped with Prolong Gold or SlowFade Gold antifading agent with DAPI (Thermo Fisher Scientific).

The following primary antibodies were used: fibrinogen (mouse IHC: 1:1000, rabbit polyclonal, gift from J. Degen, Cincinnati); GFAP (1:200, rat monoclonal, #13-0300, Thermo Fisher Scientific); GST-pi (1:200, rabbit polyclonal, #312, MBL International), ID2 (1:2000, rabbit monoclonal, #M213, CalBioreagents); MBP (1:500, #ab7349, Abcam), OLIG-2 (1:200, rabbit polyclonal, #ab9610, EMD Millipore), PDGFRB (1:100, goat polyclonal, #AF1042, R&D Systems), PLVAP (1:100, rat monoclonal, #553849, BD Pharmingen), VCAM-1 (1:50, rat monoclonal, #550547, BD Pharmingen).

Images were acquired with an Axioplan II epifluorescence microscope (Carl Zeiss) equipped with dry Plan-Neofluar objectives (10× 0.3 NA, 20× 0.5 NA, or 40× 0.75 NA), an Axiocam HRc CCD camera, and the Axiovision image analysis software; the BIOREVO BZ-9000 inverted fluorescence microscope (Keyence) equipped with a Nikon CFI 60 Series infinite optical system and Keyence imaging software; or Olympus Fluoview confocal microscope equipped with 20× NA1.0 objective. All images were processed and analyzed in ImageJ. Depending on the staining, quantification was performed on thresholded, binary images or counting of cells by researchers blind to the mouse treatment group.

### Immunoblots

Cells or tissue were lysed in RIPA lysis buffer (Thermo Fisher Scientific) supplemented with protease/phosphatase inhibitor cocktails (Calbiochem) and lysates were cleared by centrifuging at 13,000×g for 15 minutes at 4° C. Equal amounts of protein were loaded in 4%-12% bis-tris gels (Thermo Fisher Scientific) and analyzed by western blotting. Bands were visualized with HRP-conjugated secondary antibodies (Cell Signaling Technology). Densitometry was performed using ImageJ Software (NIH) with values for each band normalized to GAPDH loading controls from the same membrane. Primary antibodies were: Id2 (1:1000, rabbit monoclonal, #M213, CalBioreagents); phospho-Smad1/5 (1:1000, rabbit monoclonal, #9516, Cell Signaling Technology); GAPDH (1:1000, rabbit monoclonal, #2118, Cell Signaling Technology)

### Statistical Analyses

Statistical analyses were performed with GraphPad Prism (Version 8). Data are presented as mean±s.e.m. No statistical methods were used to predetermine sample size, but sample sizes are similar to those reported previously. Statistical significance was determined with two-sided unpaired student's t-test, or two-sided Mann-Whitney test, or a one-way or two-way analysis of variance (ANOVA) followed by Dunnett's or Tukey's post-test for multiple comparisons as indicated in the figure legends. P value≤0.05 was considered significant. Mice with similar EAE scores (≤0.5 score difference) were randomly assigned to experimental groups and each cage had animals from each treatment group to minimize confounders. The EAE clinical scoring, histopathological analysis, and quantification were done in a blinded manner. To compare clinical scores for EAE, statistical significance of the changes in the mean clinical score for each day of the EAE experiment was estimated using permutation tests.²³ The corresponding P values were estimated using 1000 permutations. In each permutation, mice were randomly permuted. In the NOD-EAE model, means of maximum scores from the last 20 days of treatment were compared between each group with a Welch's two-sample t-test.

### BIBLIOGRAPHY

1. Zhu X, Hill R A, Dietrich D, Komitova M, Suzuki R, Nishiyama A. Age-dependent fate and lineage restriction of single NG2 cells. Development. 2011; 138(4): 745-53.
2. Madisen L, Zwingman T A, Sunkin S M, et al. A robust and high-throughput Cre reporting and characterization system for the whole mouse brain. Nat Neurosci. 2010; 13(1):133-40.
3. Jung S, Aliberti J, Graemmel P, et al. Analysis of fractalkine receptor CX(3)CR1 function by targeted deletion and green fluorescent protein reporter gene insertion. Mol Cell Biol. 2000; 20(11):4106-14.
4. Mayo L, Trauger S A, Blain M, et al. Regulation of astrocyte activation by glycolipids drives chronic CNS inflammation. Nat Med. 2014; 20(10):1147-56.
5. Mendiola A S, Ryu J K, Bardehle S, et al. Transcriptional profiling and therapeutic targeting of oxidative stress in neuroinflammation. Nat Immunol. 2020; 21(5):513-524.
6. Wu Y E, Pan L, Zuo Y, Li X, Hong W. Detecting activated cell populations using single-cell RNA-seq. Neuron. 2017; 96(2):313-329 e6.
7. Robinson M D, McCarthy D J, Smyth G K. edgeR: a Bioconductor package for differential expression analysis of digital gene expression data. Bioinformatics. 2010; 26(1): 139-40.
8. Zhou Y, Zhou B, Pache L, et al. Metascape provides a biologist-oriented resource for the analysis of systems-level datasets. Nat Commun. 2019; 10(1): 1523.
9. Subramanian A, Tamayo P, Mootha V K, et al. Gene set enrichment analysis: a knowledge-based approach for interpreting genome-wide expression profiles. Proc Natl Acad Sci USA. 2005; 102(43): 15545-50.
10. Mootha V K, Lindgren C M, Eriksson K F, et al. PGC-1alpha-responsive genes involved in oxidative phosphorylation are coordinately downregulated in human diabetes. Nat Genet. 2003; 34(3):267-73.
11. Shannon P, Markiel A, Ozier O, et al. Cytoscape: a software environment for integrated models of biomolecular interaction networks. Genome Res. 2003; 13(11):2498-504.
12. Davalos D, Lee J K, Smith W B, et al. Stable in vivo imaging of densely populated glia, axons and blood vessels in the mouse spinal cord using two-photon microscopy. J Neurosci Methods. 2008; 169(1):1-7.
13. Romanelli E, Sorbara C D, Nikić I, Dagkalis A, Misgeld T, Kerschensteiner M. Cellular, subcellular and functional in vivo labeling of the spinal cord using vital dyes. Nat Protoc. 2013; 8(3):481-490.
14. Wilke S A, Antonios J K, Bushong E A, et al. Deconstructing complexity: serial block-face electron microscopic analysis of the hippocampal mossy fiber synapse. J Neurosci. 2013; 33(2):507-22.
15. Walton J. Lead aspartate, an en bloc contrast stain particularly useful for ultrastructural enzymology. J Histochem Cytochem. 1979; 27(10): 1337-42.
16. Bushong E A, Johnson D D, Jr., Kim K Y, et al. X-ray microscopy as an approach to increasing accuracy and efficiency of serial block-face imaging for correlated light and electron microscopy of biological specimens. Microsc Microanal. 2015; 21(1):231-8.
17. Kremer J R, Mastronarde D N, McIntosh J R. Computer visualization of three-dimensional image data using IMOD. J Struct Biol. 1996; 116(1):71-6.
18. Petersen M A, Ryu J K, Chang K J, et al. Fibrinogen activates BMP signaling in oligodendrocyte progenitor cells and inhibits remyelination after vascular damage. Neuron. 2017; 96(5): 1003-1012 e7.
19. Mei F, Fancy S P J, Shen Y A, et al. Micropillar arrays as a high-throughput screening platform for therapeutics in multiple sclerosis. Nat Med. 2014; 20(8):954-960.
20. Najm F J, Madhavan M, Zaremba A, et al. Drug-based modulation of endogenous stem cells promotes functional remyelination in vivo. Nature. 2015; 522(7555):216-20.
21. Mei F, Mayoral S R, Nobuta H, et al. Identification of the kappa-opioid receptor as a therapeutic target for oligodendrocyte remyelination. J Neurosci. 2016; 36(30):7925-35.
22. Fancy S P, Harrington E P, Yuen T J, et al. Axin2 as regulatory and therapeutic target in newborn brain injury and remyelination. Nat Neurosci. 2011; 14(8): 1009-16.
23. Ryu J K, Rafalski V A, Meyer-Franke A, et al. Fibrin-targeting immunotherapy protects against neuroinflammation and neurodegeneration. Nat Immunol. 2018; 19(11): 1212-1223.

### Results

NG2 Cells Cluster Perivascularly at Sites of Fibrinogen Deposition with Limited Remyelination in Chronic Neuroinflammation

NG2 cells, also referred to as OPCs, are progenitor cells in the adult CNS closely associated with the vasculature with unique potential to promote remyelination (Dimou and Gallo, 2015). To study NG2 cells and neurovascular dysfunction in neuroinflammation, NG2-CreER^{TM}:Rosa^{tdTomato/+}:Cx3cr1^{GFP/+} mice were generated. In vivo 2P imaging and transcriptomic profiling of NG2 cells and microglia during chronic experimental autoimmune encephalomyelitis (EAE) induced by the epitope of amino acids 35-55 of myelin oligodendrocyte glycoprotein (MOG) (‘MOG₃₅₋₅₅ EAE’) were performed (Supplementary FIG. 1). Extravasation of 70 kilodalton Oregon Green Dextran was used as a marker of acute BBB leakage, and fibrinogen immunohistology as a marker of chronic BBB leakage and local coagulation. At peak EAE, perivascular clusters consisted primarily of microglia, and NG2 cells were evenly distributed in the spinal cord parenchyma (FIG. 1A, Supplementary FIG. 2A). However, in chronic EAE, perivascular clusters also consisted of NG2 cells, with more than ˜80% of NG2 cell clusters located at or within 30 μm of a blood vessel (FIG. 1A, Supplementary FIG. 2B). NG2^{tdTomato+} cells in the clusters had glial-like morphology characterized by multiple branched processes in the spinal cord parenchyma, distinguishable from NG2^{tdTomato+} pericytes with elongated processes along the blood vessel wall (Supplementary FIG. 2C). VCAM1, a marker of endothelial activation (Lengfeld et al., 2017), and PLVAP, a marker of endothelial fenestrae in leaky CNS vessels (Niu et al., 2019), were increased in peak and chronic EAE white matter (Supplementary FIG. 3A, B), suggesting disruption of neurovascular homeostasis. Fibrinogen deposition is a prominent feature of neurovascular pathology in EAE, necessary for disease pathogenesis (Adams et al., 2007; Davalos et al., 2012; Ryu et al., 2018). While acute dextran leakage was highest at peak EAE, fibrinogen deposition increased over time and was highest during chronic EAE (FIG. 1B), suggesting persistent fibrinogen deposition even when active BBB disruption declined. In chronic EAE, NG2 clusters aggregated perivascularly only at sites of fibrinogen deposition (FIG. 1C, Supplementary FIG. 4A), and often co-localized with microglial clusters (FIG. 1A, Supplementary FIG. 2A). These results suggest dynamic glial remodeling of the neurovascular interface at sites of fibrinogen deposition during neuroinflammation.

To assess myelin within perivascular NG2 clusters using in vivo 2P imaging, MitoTracker Deep Red, a mitochondrial dye that also labels myelin when used at higher concentrations (Romanelli et al., 2013), was applied. Significant myelin disruption, characterized by blebbing of myelin sheaths, was present near NG2 clusters, whereas normal-appearing myelin sheaths appeared at sites without clusters (FIG. 1D, Supplementary FIG. 4B). To study myelin ultrastructure, a co-registration technique was developed to correlate 2P-imaged volume with three-dimensional serial block face electron microscopy (SBEM) using microcomputed tomography (FIG. 1E). Using this technique, SBEM images were collected at the exact same areas of perivascular NG2 clusters in EAE mice imaged by in vivo 2P microscopy. Inflamed veins with endothelial activation, attachment of leukocytes at the endothelial surface, perivascular astrogliosis, and inflammation, in part with debris-containing macrophages were observed (FIG. 1Fi, Gi, Supplementary FIG. 3C). In the parenchymal lesions we found two distinct patterns: the first was characterized by cell infiltration of elongated cells with low cell density, some of which contained osmiophilic degradation products. In these areas, axons were predominantly demyelinated, and remyelination was sparse (FIG. 1Fii, Gi). In other areas, there were more dense clusters of small cells with small rims of perinuclear cytoplasm containing some mitochondria, but few other organelles, which were reminiscent of NG2 cells (FIG. 1Gii). Remyelinated axons were closely adjacent to these cell clusters, while in areas distant from the clusters, axons were demyelinated (FIG. 1Fiii, Gii). Away from perivascular NG2 cells, normal-appearing perivascular CNS tissue, astrocytic glia limitans, and axons with normal myelin thickness were observed (FIG. 1Fiv). These results suggest that perivascular NG2 clusters are associated with inflammation, gliosis, frank demyelination and limited remyelination.

### Transcriptomic Profiling of NG2 Cells in EAE Reveals Suppression of Vascular Homeostasis and Anticoagulation Pathways

To study the transcriptomic changes in NG2 cells in chronic EAE, RNA-seq was performed on NG2^{tdTomato+} cells collected from the spinal cords of MOG₃₅₋₅₅ EAE mice or healthy controls (Supplementary FIG. 3A). A total of 1,241 differentially expressed genes (DEGs) (FDR<0.05; ±1 log₂ fold change) were identified in the setting of chronic EAE compared to control, of which 738 were downregulated (60%) and 503 upregulated (40%) (FIG. 2A). Unsupervised gene clustering analysis identified 9 distinct gene clusters (FIG. 2B). Gene ontology (GO) analysis revealed that chronic EAE activated inflammatory and antigen-presentation genes in clusters 1-4, including the GO pathway terms “Positive regulation of acute inflammatory response,” “Positive regulation of T cell mediated cytotoxicity,” “Antigen processing and presentation,” and “Cellular response to interferon-beta” (FIG. 2B). Canonical antigen presentation genes, such as Cd74, H2-dma, and B2m, were significantly upregulated in EAE (FIG. 2B), consistent with reports suggesting immune-like functions of OL lineage cells in disease (Falcao et al., 2018; Kirby et al., 2019). Interestingly, GO analysis of downregulated gene clusters 5-9 revealed pathways related to vascular and BBB homeostasis, such as “Angiogenesis,” “Regulation of Wnt signaling pathway,” “Vasculogenesis,” “Blood vessel development,” and “Cell junction organization” (FIG. 2B). In accordance, gene networks involved in blood vessel maintenance, wound healing and coagulation, and tight junctions were globally repressed in EAE (FIG. 2C). Gene set enrichment analysis (GSEA) of DEGs identified the top two downregulated gene sets as “Regulation of cell junction assembly” (normalized enrichment score (NES) 1.7, p<0.01) and “Negative regulation of coagulation” (NES 1.7, p<0.01) (FIG. 2D). Expression of tissue factor pathway inhibitor (Tfpi), a primary inhibitor of blood coagulation and fibrin formation (Wood et al., 2014), was significantly reduced in NG2 cells in EAE. As the NG2^{tdTomato+} population includes OPC and pericyte lineages, we isolated PDGFRα⁺ OPCs and PDGFRβ⁺ pericytes from the spinal cords of MOG₃₅₋₅₅-EAE mice or healthy controls (Supplementary FIG. 3B) and labeled cell surface major histocompatibility complex class II (MHCII) and TFPI to assess the antigen presentation and anticoagulation pathways, respectively. Consistent with our bulk-RNAseq and prior studies (Kirby et al., 2019), MHCII was increased in PDGFRα⁺ OPCs in EAE (Supplementary FIG. 3C). TFPI was expressed by OPCs but not pericytes in healthy controls and was significantly repressed in EAE (FIG. 2E, F). Overall, these results identify dysregulation of antigen presentation, coagulation, and vascular homeostasis pathways in OPCs in chronic neuroinflammation.

### Promyelinating Compounds do not Overcome Fibrinogen Extrinsic Inhibition of OPC Differentiation

OPCs can differentiate to myelinating OLs or astrocyte-like cells in response to extrinsic signals found in multiple sclerosis lesions like fibrinogen or BMPs (Mabie et al., 1997; Petersen et al., 2017; Hackett et al., 2018). We developed the OPC-X-screen, a medium-throughput, high-content imaging assay to identify compounds that in the presence of extrinsic inhibitors promote OPC differentiation to mature MBP⁺ OLs and decrease the OPC fate-switch to GFAP⁺ astrocytes (FIG. 3A). In the OPC-X assay, fibrinogen decreased MBP⁺ mature OLs and increased GFAP⁺ astrocyte-like cells by ˜60% as compared to controls (FIG. 3B-D). Seven compounds—benztropine, clemastine, quetiapine, miconazole, clobetasol, (±)U-50488, and XAV-939—have been previously identified to promote intrinsic pathways of OPC differentiation (Fancy et al., 2011; Mei et al., 2014; Najm et al., 2015; Mei et al., 2016). However, these promyelinating compounds did not overcome extrinsic inhibition of OPC differentiation by fibrinogen (FIG. 3B-D). In contrast, the BMP receptor inhibitor DMH1 (Hao et al., 2010) rescued the inhibitory effects of fibrinogen and restored OPC differentiation to mature OLs to control levels (FIG. 3B-D). Cell-fate switch of OPCs to GFAP⁺ cells by fibrinogen was also abolished by DMH1 (FIG. 3D). Clemastine, a muscarinic receptor antagonist, promotes the remyelinating potential of OPCs and is currently in clinical trials for multiple sclerosis (Mei et al., 2014; Green et al., 2017). While clemastine increased the number of MBP⁺ cells in control conditions as expected, it did not enhance OPC differentiation to mature OLs in the presence of fibrinogen (Supplementary FIG. 4). Clemastine did not block fibrinogen-induced phosphorylation of the BMP signal transducers SMAD1/5 or expression of the BMP target protein ID2 (FIG. 3E). In contrast, DMH1 blocked fibrinogen induced SMAD1/5 phosphorylation and ID2 expression (FIG. 3E). Thus, previously identified compounds promoting OPC differentiation may not overcome extrinsic inhibition signaling pathways at sites of vascular damage.

### Therapeutic Effects of Type I BMP Receptor Inhibition in Neuroinflammation

BMP expression and downstream receptor signaling is increased in human multiple sclerosis lesions (Costa et al., 2019; Harnisch et al., 2019). The BMP target protein ID2 is also increased in lesions with extensive fibrinogen deposition (Petersen et al., 2017). The finding that DMH1 effectively blocked fibrinogen-induced BMP receptor activation and restored OPC differentiation in vitro (FIG. 3) suggested that targeting BMP signaling may promote repair in neuroinflammation. However, DMH1 is not water-soluble, which limits its use in vivo. Therefore, we tested LDN-212854, a water-soluble activin A receptor type I (ACVR1)-biased type I BMP receptor inhibitor with a molecular structure similar to DMH1 (Mohedas et al., 2013), in the OPC-X-Screen. LDN-212854 restored mature OL differentiation and blocked the formation of GFAP+ astrocytes from fibrinogen-treated OPCs in a dose-dependent manner (FIG. 3F,G).

To determine the therapeutic potential of LDN-212854, we selected two models of EAE: chronic MOG₃₅₋₅₅ EAE induced in NG2-CreER^{TM}:Rosa^{tdTomato/+} mice and progressive EAE induced in non-obese diabetic (NOD) mice by the epitope of amino acids 35-55 of MOG (‘NOD-MOG₃₅₋₅₅ EAE’) (Mayo et al., 2014). Therapeutic administration of LDN-212854 significantly improved clinical scores (FIG. 4A-D) and reduced fibrinogen deposition and demyelination in both models (FIG. 4A-D). LDN-212854 also markedly reduced perivascular NG2 clusters and myelin damage in MOG₃₅₋₅₅ EAE, as revealed by in vivo 2P imaging (FIG. 4E, F). Moreover, LDN-212854 decreased ID2 expression in NG2 cells in the EAE white matter (FIG. 4G), consistent with inhibition of BMP signaling in the NG2 cell lineage.

Since a key mechanism of fibrinogen and BMP receptor signaling is cell fate switch of OPCs to astrocytes (Mabie et al., 1997; Petersen et al., 2017), we tested whether LDN-212854 promoted OPC differentiation to myelinating cells in MOG₃₅₋₅₅ EAE. To trace the cell fate of OPCs in vivo, we induced EAE in the NG2-CreER^{TM}:Rosa^{tdTomato/+} mice, allowing tamoxifen-induced expression of tdTomato in NG2⁺ OPCs and their progeny (Petersen et al., 2017; Hackett et al., 2018). Glutathione s-transferase-pi (GST-pi) labeled mature OLs and GFAP labeled astrocytes derived from genetically-labeled tdTomato⁺ NG2⁺ OPCs. Therapeutic administration of LDN-212854 increased the proportion NG2^{tdTomato+} OPCs that differentiated into GST-pi⁺ mature OLs compared to controls, and abolished formation of OPC-derived GFAP⁺ astrocytes in NG2-CreER^{TM}:Rosa^{tdTomato/+} MOG₃₅₋₅₅ EAE mice (FIG. 4H). Collectively, these results suggest that Type I BMP receptor inhibition restores the cell fate of OPCs to mature OLs with therapeutic potential in neuroinflammatory disease with fibrinogen deposition and active BMP signaling.

### DISCUSSION

The data provided herein reveals dynamic cellular remodeling of the neurovascular niche at sites of BBB dysfunction in neuroinflammation and identifies a druggable pathway to promote myelin repair. Perhaps in neuroinflammation, perivascular NG2⁺ OPC clusters contribute to a procoagulant environment leading to excessive fibrinogen deposition, activation of BMP receptor signaling in OPCs, and extrinsic inhibition of remyelination at sites of vascular damage. This model is consistent with chronically demyelinated multiple sclerosis lesions, in which perivascular OPC clusters are localized in the active lesion borders with fibrinogen deposition, impaired fibrinolysis, BMP pathway activation, and gliosis (Petersen et al., 2017; Yates et al., 2017; Lee et al., 2018; Niu et al., 2019). Through the OPC-X-screen, we discovered that the therapeutic potential of many promyelinating drugs may be limited at sites of vascular damage and fibrinogen deposition, highlighting the unmet clinical need for therapeutic strategies to overcome extrinsic inhibition in diseases with chronic demyelination. Provided herein is the concept that inhibiting BMP pathway activation can promote myelin repair by overcoming abortive OPC differentiation at sites of neurovascular dysfunction. Thus, BMP inhibitors can expand the toolbox of promyelinating drugs and provide additional therapeutic options for patients with BBB disruption and white matter pathology.

Using in vivo 2P imaging, we found a striking transition of the perivascular glial cell composition associated with microglia and demyelination at the peak of disease, followed by the formation of perivascular NG2 clusters with limited remyelination in chronic neuroinflammation. NG2 cell clustering at sites of fibrinogen deposition suggests that OPC migration or adhesion may be altered at sites of vascular damage or that OPCs themselves may contribute to BBB disruption or local coagulation. This study suggests previously unknown functions of OPCs in the expression of genes regulating coagulation. TFPI, a potent inhibitor of coagulation factor X and tissue factor-mediated coagulation (Wood et al., 2014), was expressed in OPCs and repressed by chronic neuroinflammation. Interestingly, multiple sclerosis patients have alterations in hemostasis biomarkers including TFPI (Ziliotto et al., 2019), suggesting an imbalance in anti- and procoagulant pathways in neuroinflammatory disease. Prooxidant microglia may also contribute to the procoagulant milieu in the lesion microenvironment through expression of coagulation proteins such as coagulation factor X (Mendiola et al., 2020). Thus, transcriptional changes at the neurovascular interface may establish a local procoagulant environment that contributes to the excessive or persistent deposition of fibrin observed in many neurological diseases (Petersen et al., 2018). Therapeutic strategies to target the NG2 cell-vascular-fibrinogen axis or downstream fibrinogen signaling can provide a therapeutic avenue to overcome extrinsic inhibition in the neuroinflammatory lesion environment.

The study suggests that promyelinating drugs differentially suppress signaling pathways activated by extrinsic inhibitors in the lesion environment. Indeed, clemastine did not inhibit SMAD1/5 phosphorylation, a key pathway downstream of BMP receptor activation, or rescue OPC cell fate switch to astrocytes. Fibrinogen, in addition to activating BMP receptor signaling in OPCs, stimulates CSPG production from astrocytes and is a carrier for transforming growth factor-beta (TGF-β) (Schachtrup et al., 2010). CSPGs inhibit remyelination in part through activation of the protein tyrosine phosphatase sigma receptor in OPCs (Pendleton et al., 2013). Age-related loss of OPC function may occur in response to TGF-β signaling or increased stiffness in the OPC niche, with subsequent signaling through the mechanoresponsive ion channel Piezol (Baror et al., 2019; Segel et al., 2019). Therefore, assays that better recapitulate the inhibitory lesion environment and downstream signaling are needed to improve selection of drugs that can increase remyelination in inflammatory lesions with gliosis, vascular damage and BBB disruption. Furthermore, the choice of promyelinating drug in the clinic may need to take into account its efficacy within the extrinsic inhibitory milieu in patients with demyelinating neurological diseases. Targeting multiple inhibitory pathways with combinations of drugs may have additive or synergistic effects on remyelination and could provide an avenue to maximize the therapeutic benefit of promyelinating compounds in an inhibitory lesion environment.

Therapeutic fibrinogen depletion by anticoagulants can suppress neuroinflammation and promote myelin regeneration (Akassoglou et al., 2002; Petersen et al., 2017), but hemorrhagic complications may limit the clinical utility of this approach. The instant study identifies LDN-212854, an ACVR1-biased BMP receptor inhibitor, as a potential therapeutic agent for chronic neuroinflammation. Activation of fibrinogen and BMP signaling in the injured perivascular niche directs OPC cell fate towards astrocytes rather than remyelinating OLs (Petersen et al., 2017; Baror et al., 2019), which may contribute to pathologic gliosis at sites of vascular damage. LDN-212854 increased myelinating OLs and eliminated OPC differentiation to astrocytes. LDN-212854 was well-tolerated at the doses used in the study, but human toxicity data is limited. Clinical use of ACVR1-selective BMP inhibitors has gained recent attention for the treatment of fibrodysplasia ossificans progressive, a rare disorder with overactive BMP signaling resulting in heterotopic ossification and myelin abnormalities (Kan et al., 2012). LDN-212854 and other safe ACVR1-selective inhibitors may be a therapeutic option for neurological diseases with BBB disruption and myelin abnormalities including multiple sclerosis, Alzheimer disease, neonatal brain injury, and traumatic brain injury.

### BIBLIOGRAPHY

Adams R A, Bauer J, Flick M J, Sikorski S L, Nuriel T, Lassmann H, et al. The fibrin-derived gamma377-395 peptide inhibits microglia activation and suppresses relapsing paralysis in central nervous system autoimmune disease. J Exp Med 2007; 204(3): 571-82.
Akassoglou K, Yu W-M, Akpinar P, Strickland S. Fibrin inhibits peripheral nerve regeneration by arresting Schwann cell differentiation. Neuron 2002; 33: 861-75.
Back S A, Gan X, Li Y, Rosenberg P A, Volpe J J. Maturation-dependent vulnerability of oligodendrocytes to oxidative stress-induced death caused by glutathione depletion. J Neurosci 1998; 18(16): 6241-53.
Baror R, Neumann B, Segel M, Chalut K J, Fancy S P J, Schafer D P, et al. Transforming growth factor-beta renders ageing microglia inhibitory to oligodendrocyte generation by CNS progenitors. Glia 2019; 67(7): 1374-84.
Costa C, Eixarch H, Martinez-Saez E, Calvo-Barreiro L, Calucho M, Castro Z, et al. Expression of bone morphogenetic proteins in multiple sclerosis lesions. Am J Pathol 2019; 189(3): 665-76.
Davalos D, Ryu J K, Merlini M, Baeten K M, Le Moan N, Petersen M A, et al. Fibrinogen-induced perivascular microglial clustering is required for the development of axonal damage in neuroinflammation. Nat Commun 2012; 3: 1227.
Deshmukh V A, Tardif V, Lyssiotis C A, Green C C, Kerman B, Kim H J, et al. A regenerative approach to the treatment of multiple sclerosis. Nature 2013; 502(7471): 327-32.
Dimou L, Gallo V. NG2-glia and their functions in the central nervous system. Glia 2015; 63(8): 1429-51.
Falcao A M, van Bruggen D, Marques S, Meijer M, Jakel S, Agirre E, et al. Disease-specific oligodendrocyte lineage cells arise in multiple sclerosis. Nat Med 2018; 24(12): 1837-44.
Fancy S P, Harrington E P, Yuen T J, Silbereis J C, Zhao C, Baranzini S E, et al. Axin2 as regulatory and therapeutic target in newborn brain injury and remyelination. Nat Neurosci 2011; 14(8): 1009-16.
Forbes T A, Gallo V. All wrapped up: environmental effects on myelination. Trends Neurosci 2017; 40(9): 572-87.
Franklin R J M, Ffrench-Constant C. Regenerating CNS myelin—from mechanisms to experimental medicines. Nat Rev Neurosci 2017; 18(12): 753-69.
Green A J, Gelfand J M, Cree B A, Bevan C, Boscardin W J, Mei F, et al. Clemastine fumarate as a remyelinating therapy for multiple sclerosis (ReBUILD): a randomised, controlled, double-blind, crossover trial. Lancet 2017; 390(10111): 2481-9.
Hackett A R, Yahn S L, Lyapichev K, Dajnoki A, Lee D H, Rodriguez M, et al. Injury type-dependent differentiation of NG2 glia into heterogeneous astrocytes. Exp Neurol 2018; 308: 72-9.
Hao J, Ho J N, Lewis J A, Karim K A, Daniels R N, Gentry P R, et al. In vivo structure-activity relationship study of dorsomorphin analogues identifies selective VEGF and BMP inhibitors. ACS Chem Biol 2010; 5(2): 245-53.
Harnisch K, Teuber-Hanselmann S, Macha N, Mairinger F, Fritsche L, Soub D, et al. Myelination in multiple sclerosis lesions is associated with regulation of bone morphogenetic protein 4 and its antagonist noggin. Int J Mol Sci 2019; 20(1).
Kan L, Kitterman J A, Procissi D, Chakkalakal S, Peng C Y, McGuire T L, et al. CNS demyelination in fibrodysplasia ossificans progressiva. J Neurol 2012; 259(12): 2644-55.
Keough M B, Rogers J A, Zhang P, Jensen S K, Stephenson E L, Chen T, et al. An inhibitor of chondroitin sulfate proteoglycan synthesis promotes central nervous system remyelination. Nat Commun 2016; 7: 11312.
Kirby L, Jin J, Cardona J G, Smith M D, Martin K A, Wang J, et al. Oligodendrocyte precursor cells present antigen and are cytotoxic targets in inflammatory demyelination. Nat Commun 2019; 10(1): 3887.
Lee N J, Ha S K, Sati P, Absinta M, Luciano N J, Lefeuvre J A, et al. Spatiotemporal distribution of fibrinogen in marmoset and human inflammatory demyelination. Brain 2018; 141(6): 1637-49.
Lengfeld J E, Lutz S E, Smith J R, Diaconu C, Scott C, Kofman S B, et al. Endothelial Wnt/beta-catenin signaling reduces immune cell infiltration in multiple sclerosis. Proc Natl Acad Sci USA 2017; 114(7): E1168-E77.
Mabie P C, Mehler M F, Marmur R, Papavasiliou A, Song Q, Kessler J A. Bone morphogenetic proteins induce astroglial differentiation of oligodendroglial-astroglial progenitor cells. J Neurosci 1997; 17(11): 4112-20.
Magliozzi R, Hametner S, Facchiano F, Marastoni D, Rossi S, Castellaro M, et al. Iron homeostasis, complement, and coagulation cascade as CSF signature of cortical lesions in early multiple sclerosis. Ann Clin Transl Neurol 2019; 6(11): 2150-63.
Mayo L, Trauger S A, Blain M, Nadeau M, Patel B, Alvarez J I, et al. Regulation of astrocyte activation by glycolipids drives chronic CNS inflammation. Nat Med 2014; 20(10): 1147-56.
Mei F, Fancy S P J, Shen Y A, Niu J, Zhao C, Presley B, et al. Micropillar arrays as a high-throughput screening platform for therapeutics in multiple sclerosis. Nat Med 2014; 20(8): 954-60.
Mei F, Mayoral S R, Nobuta H, Wang F, Desponts C, Lorrain D S, et al. Identification of the Kappa-Opioid Receptor as a Therapeutic Target for Oligodendrocyte Remyelination. J Neurosci 2016; 36(30): 7925-35.
Mendiola A S, Ryu J K, Bardehle S, Meyer-Franke A, Ang K K, Wilson C, et al. Transcriptional profiling and therapeutic targeting of oxidative stress in neuroinflammation. Nat Immunol 2020; 21(5): 513-24.
Miron V E, Boyd A, Zhao J W, Yuen T J, Ruckh J M, Shadrach J L, et al. M2 microglia and macrophages drive oligodendrocyte differentiation during CNS remyelination. Nat Neurosci 2013; 16(9): 1211-8.
Mohedas A H, Xing X, Armstrong K A, Bullock A N, Cuny G D, Yu P B. Development of an ALK2-biased BMP type I receptor kinase inhibitor. ACS Chem Biol 2013; 8(6): 1291-302.
Najm F J, Madhavan M, Zaremba A, Shick E, Karl R T, Factor D C, et al. Drug-based modulation of endogenous stem cells promotes functional remyelination in vivo. Nature 2015; 522(7555): 216-20.
Neumann B, Baror R, Zhao C, Segel M, Dietmann S, Rawji K S, et al. Metformin restores CNS remyelination capacity by rejuvenating aged stem cells. Cell Stem Cell 2019; 25(4): 473-85 e8.
Niu J, Tsai H H, Hoi K K, Huang N, Yu G, Kim K, et al. Aberrant oligodendroglial-vascular interactions disrupt the blood-brain barrier, triggering CNS inflammation. Nat Neurosci 2019; 22(5): 709-18.
Pendleton J C, Shamblott M J, Gary D S, Belegu V, Hurtado A, Malone M L, et al. Chondroitin sulfate proteoglycans inhibit oligodendrocyte myelination through PTPsigma. Exp Neurol 2013; 247: 113-21.
Petersen M A, Ryu J K, Akassoglou K. Fibrinogen in neurological diseases: mechanisms, imaging and therapeutics. Nat Rev Neurosci 2018; 19(5): 283-301.
Petersen M A, Ryu J K, Chang K J, Etxeberria A, Bardehle S, Mendiola A S, et al. Fibrinogen activates BMP signaling in oligodendrocyte progenitor cells and inhibits remyelination after vascular damage. Neuron 2017; 96(5): 1003-12 e7.
Pous L, Deshpande S S, Nath S, Mezey S, Malik S C, Schildge S, et al. Fibrinogen induces neural stem cell differentiation into astrocytes in the subventricular zone via BMP signaling. Nat Commun 2020; 11(1): 630.
Reich D S, Lucchinetti C F, Calabresi P A. Multiple Sclerosis. N Engl J Med 2018; 378(2): 169-80.
Romanelli E, Sorbara C D, Nikić I, Dagkalis A, Misgeld T, Kerschensteiner M. Cellular, subcellular and functional in vivo labeling of the spinal cord using vital dyes. Nat Protoc 2013; 8(3): 481-90.
Ryu J K, Petersen M A, Murray S G, Baeten K M, Meyer-Franke A, Chan J P, et al. Blood coagulation protein fibrinogen promotes autoimmunity and demyelination via chemokine release and antigen presentation. Nat Commun 2015; 6: 8164.
Ryu J K, Rafalski V A, Meyer-Franke A, Adams R A, Poda S B, Rios Coronado P E, et al. Fibrintargeting immunotherapy protects against neuroinflammation and neurodegeneration. Nat Immunol 2018; 19(11): 1212-23.
Schachtrup C, Ryu J K, Helmrick M J, Vagena E, Galanakis D K, Degen J L, et al. Fibrinogen triggers astrocyte scar formation by promoting the availability of active TGF-beta after vascular damage. J Neurosci 2010; 30(17): 5843-54.
Segel M, Neumann B, Hill M F E, Weber I P, Viscomi C, Zhao C, et al. Niche stiffness underlies the ageing of central nervous system progenitor cells. Nature 2019; 573(7772): 130-4.
Starost L, Lindner M, Herold M, Xu Y K T, Drexler H C A, Hess K, et al. Extrinsic immune cell derived, but not intrinsic oligodendroglial factors contribute to oligodendroglial differentiation block in multiple sclerosis. Acta Neuropathol 2020; 140(5): 715-36.
Vos C M, Geurts J J, Montagne L, van Haastert E S, Bo L, van der Valk P, et al. Blood-brain barrier alterations in both focal and diffuse abnormalities on postmortem MRI in multiple sclerosis. Neurobiol Dis 2005; 20(3): 953-60.
Wood J P, Ellery P E, Maroney S A, Mast A E. Biology of tissue factor pathway inhibitor. Blood 2014; 123(19): 2934-43.
Yates R L, Esiri M M, Palace J, Jacobs B, Perera R, DeLuca G C. Fibrin(ogen) and neurodegeneration in the progressive multiple sclerosis cortex. Ann Neurol 2017; 82(2): 259-70.
Ziliotto N, Bernardi F, Jakimovski D, Zivadinov R. Coagulation pathways in neurological diseases: multiple sclerosis. Front Neurol 2019; 10: 409.

All publications, nucleotide and amino acid sequence identified by their accession nos., patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The specific methods and compositions described herein are representative of embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims and statements of the invention.

1. A high-throughput, high-content assay to screen for an agent which overcomes remyelination inhibition by an extrinsic inhibitor comprising:
a) contacting oligodendrocyte progenitor cells (OPCs) with an extrinsic inhibitor and a test agent and
b) obtaining two readouts in a single assay to detect/quantify the presence of: 1) MBP+ myelinating oligodendrocytes (OLs) and 2) GFAP+ astrocytes,
wherein an increase in OLs and a decrease in GFAP+ astrocytes as compared to a control OPCs only contacted with the extrinsic inhibitor, indicates the agent overcame inhibition of remyelination by an extrinsic inhibitor.

2. The assay of claim 1, wherein the extrinsic inhibitor is an antibody, a compound, a small molecule, a peptide and/or a nucleic acid.

3. The assay of claim 1, wherein the extrinsic inhibitor is an inflammatory molecule.

4. The assay of claim 1, wherein the extrinsic inhibitor is fibrinogen.

5. The assay of claim 4, wherein fibrinogen is present at a physiological level.

6. The assay of claim 4, wherein fibrinogen is added at a concentration of least 2.5 mg/ml.

7. The assay of claim 1, wherein the OPCs are primary OPCs.

8. The assay of claim 7, wherein the primary OPCs are cultured in proliferation medium for 1-6 days prior to a).

9. The assay of claim 8, wherein the proliferation medium comprises PDGF-AA and NT3.

10. The assay of claim 8, wherein the OPCs are detached from culture dish proteolytically and/or collagenolytically and then plated into fresh culture dishes.

11. The assay of claim 10, wherein the OPCs are plated at 5×10³ cells/well of a 96 well plate or 1×10³ cells/well of a 384 well plate prior to a).

12. The assay of claim 11, wherein the plated OPCs are cultured for up to 24 hours prior to a).

13. The assay of claim 1, wherein the OPCs are cultured in step a) for 1-6 days so that the OPCs can differentiate prior to b).

14. The assay of claim 13, where the OPCs are cultured for 3 days.

15. The assay of claim 13, wherein the differentiation medium comprises CNTF triiodothyronine (T3) and PDGF-AA.

16. The assay of claim 1, wherein after a), the cells are contacted with antibodies against MBP (oligodendrocytes) and antibodies against GFAP (astrocytes).

17. The assay of claim 16, wherein the antibodies are labeled directed or indirectly with a detectable label and images of the cells with labels are obtained.

18. The assay of claim 17, wherein automated images are obtained.

19. The assay of claim 17, wherein at least about 80% of the culture vessel (e.g., well) is imaged.

20. The assay claim 1, wherein automated quantification of MBP+ and GFAP+ is employed.

21. A high-throughput, high-content assay to screen for an agent which overcomes inhibition of extrinsic inhibitor comprising:
a) contacting a cell with an extrinsic inhibitor and a test agent and
b) detecting and/or quantifying the cells response to the test agent,
wherein a response by the cells that is different as compared to a control in which the cells are only contacted with the extrinsic inhibitor, indicates the agent was able to overcome the extrinsic inhibitor.

22. The assay of claim 21, wherein the extrinsic inhibitor is selected from the group consisting of chondroitin sulfate proteoglycan, hyaluronan, fibronectin aggregate, myelin debris, inflammatory cytokine (e.g., soluble TNF-alpha or Interferon-gamma), bone morphogenetic protein, endothelin-1, semaphorin, environmental toxin and alcohol, tobacco or illicit or recreational drugs.

23. The assay of claim 21, wherein the cell is selected from the group consisting of stem or progenitor cells including neural stem and/or progenitor cells (adult and/or fetal/neonatal), radial glial cells (adult and/or fetal/neonatal), cerebellar granule neuron progenitor cells, neural crest stem/progenitor cells, vascular/endothelial stem/progenitor cells, organ stem/progenitor cells (e.g., cardiac, liver, lung, kidney, skeletal muscle, skin, bone, retinal), mesenchymal stem/progenitor cells, placental stem/progenitor cells, embryonic stem cells, induced pluripotent stem cells (or cells derived from ESCs/iPSCs), and cancer/tumor-associated cells/stem cells.

24. The assay of claim 23, wherein the neural progenitors are oligodendrocyte progenitor cells (OPCs).


SEQUENCE LISTING (1 sequence)

SEQ ID NO: 1 - protein, 21 aa, Artificial Sequence
  A synthetic sequence
MEVGWYRSPFSRVVHLYRNGK